(19) United States
(12) Patent Application Publication
(10) Pub. No.: US 2009/0088731 A1
(43) Pub. Date: Apr. 2, 2009

(54) Infusion Device Menu Structure and Method of Using the Same

(75) Inventors: Arthur Campbell, Stevenson Ranch, CA (US); Deborah Ruppert, Los Angeles, CA (US); Mike Charles Vallet Tolle, Van Nuys, CA (US)

Correspondence Address: MEDTRONIC MINIMED INC., 18000 DEVONSHIRE STREET, NORTHRIDGE, CA, 91325-1219, US

(73) Assignee: Medtronic MiniMed, Inc., Northridge, CA (US)

(21) Appl. No.: 12/333,616

(22) Filed: Dec. 12, 2008

Related U.S. Application Data
(62) Division of application No. 10/996,136, filed on Nov. 22, 2004.
Application No. 10/996,136 is a division of application No. 09/784,949, filed on Feb. 15, 2001, now abandoned.
(60) Provisional application No. 60/182,929, filed on Feb. 16, 2000.

(51) Int. Cl. A61K 9/22

(52) U.S. Cl. 604/890.1

(57) ABSTRACT

A portable infusion system that is programmable by an individual for delivering fluid from a reservoir into a user includes a drive mechanism, an input device, a processor, and a display. The drive mechanism forces the fluid out of the reservoir, and the input device accepts one or more inputs. The processor uses one or more of the one or more inputs to control the drive mechanism. The display receives information from the processor and visually displays one or more screens containing the information. At least one of the one or more screens includes a menu with at least two menu items, and the input device is used to select one menu item from amongst the at least two menu items.

### RELATED APPLICATIONS

This application claims priority on U.S. Provisional Application Ser. No. 60/182,929 filed Feb. 16, 2000, entitled “Improved Infusion Device Menu Structure And Method Of Using The Same”, and is related to U.S. patent application Ser. No. 09/334,858 filed on Jun. 17, 1999, both of which are specifically incorporated by reference herein.

### FIELD OF THE INVENTION

This invention relates to infusion devices and, in particular embodiments, to a medication infusion device that includes a menu structure that is used to control the infusion device.

### BACKGROUND OF THE INVENTION

Insulin must be provided to people with Type I diabetes and to many people with Type II diabetes. Traditionally, insulin is injected with a syringe, since it cannot be taken orally. More recently, use of external infusion pump therapy has been increasing, especially for delivering insulin for diabetics. Typically, an external infusion pump is worn on a belt, in a pocket, or the like, with the insulin delivered from a reservoir in the pump to a body via a catheter with a percutaneous needle or cannula placed in the subcutaneous tissue. For example, as of 1995, less than 5% of Type I diabetics in the United States were using pump therapy. Presently about 10% of the currently over 900,000 Type I diabetics in the U.S. are using insulin pump therapy, and the percentage is now growing at an absolute rate of over 2% each year. Moreover, the number of Type I diabetics is growing at 3% or more per year. In addition, growing numbers of insulin using Type II diabetics are also using external insulin infusion pumps. Physicians have recognized that continuous infusion provides greater control of a diabetic's condition, and are also increasingly prescribing it for patients. In addition, medication pump therapy is becoming more important for the treatment and control of other medical conditions, such as pulmonary hypertension, HIV, and cancer. Although offering control, pump therapy can suffer from several complications that make use of a pump less desirable for the user.

One drawback is the inability to easily program the infusion device and to view various features and data contained in the infusion pump, particularly when the user is a patient and not a doctor or a nurse. Many users must remember a specific series of keystrokes to find the feature or data. For instance, a user may need to scroll through several different programming schemes to find the desired feature. If the user is unsure of what keystrokes to use, they may have to search for a long time. Also, if they inadvertently press the wrong key or go past the desired screen, they may have to re-key all or part of the sequence of the keys to reach the desired feature or data.

### SUMMARY OF THE DISCLOSURE

It is an object of an embodiment of the present invention to provide an improved portable infusion device, which obviates for practical purposes, the above-mentioned limitations.

According to an embodiment of the invention, a portable infusion system that is programmable by an individual for delivering fluid from a reservoir into a user includes a drive mechanism, an input device, a processor, and a display. The drive mechanism forces the fluid out of the reservoir, and the input device accepts one or more inputs. The processor uses one or more of the one or more inputs to control the drive mechanism. The display receives information from the processor and visually displays one or more screens containing the information. At least one of the one or more screens includes a menu with at least two menu items, and the input device is used to select one menu item from amongst the at least two menu items.

In particular embodiments, one of the at least two menu items is highlighted when the menu is displayed. And the one of at least two menu items that is highlighted when the menu is displayed is dependent on a function that the infusion system is performing when the menu is displayed. In further embodiments, selection of at least one of the at least two menu items causes the drive mechanism to reverse direction. And in other embodiments, selection of at least one of the at least two menu items causes the infusion system to begin a selftest.

In another embodiment, at least one of the one or more screens is a status screen. In still other embodiments, the one or more screens includes one or more set screens, one or more select screens, or one or more confirmation screens. The one or more set screens may include a maximum basal rate screen or a maximum bolus screen. The one or more select screens may include a screen to select an insulin type, a screen to select a reservoir type, a screen to select a therapy, and/or a screen to select a language. Preferably, a numeric value displayed in at least one screen has a number to the right of a decimal point that is formatted differently than a number to the left of the decimal point.

In preferred embodiments, the processor runs energy management software that changes the display to a Blank Screen after a Time-Out delay has expired. Furthermore, the infusion device includes a means to store a maximum bolus, a maximum basal rate, and/or one or more basal profiles that are programmable using the input device. The maximum bolus limits the maximum units of fluid that can be delivered in a single bolus, and the maximum basal rate limits the maximum rate that units of fluid that can be delivered during a basal fluid delivery.

Particular embodiments include one or more alarm types, a means to store an insulin type, and/or a means to store a reservoir type, each of which are programmable using the input device. Preferred embodiments may include a means to reset control parameters to factory default values, or to values set by a health care professional. In other embodiments, the invention includes an alarm wherein the alarm intensity changes with time.

Preferred embodiments include a housing that houses the reservoir, the drive mechanism, the input device, the processor, and the display. Additionally, particular embodiments include an infusion set and tubing having a first end and a second end, wherein the first end of the tubing is connected to the reservoir and the second end of the tubing is connected to the infusion set. When tubing is included, a manual prime or a fixed prime may be used to fill the tubing with fluid from the reservoir. Preferably, information is shown on the display screen to guide the individual through the steps to prime the infusion system.

Some embodiments of the present invention include a communication device. Preferably, selection of at least one of the at least two menu items causes the display to show a screen that allows an individual to signify the identity of a device, which thereby configures the infusion system to accept communication from the device.

In preferred embodiments, the input device includes a keypad with one or more keys. And, when the infusion system is suspended from delivering fluid, fluid delivery is resumable with two or less keystrokes independent of the screen being displayed. In particular embodiments, the one or more keys may include an ACT key, and pressing the ACT key enters a selection or a value into the processor and causes the display to exit a screen that displayed the selection or value. Furthermore, the one or more keys may include an Esc key, and pressing the Esc key causes the display to exit a screen without entering a new selection or a new value into the processor. Alternatively, pressing the Esc key causes the display to exit a currently displayed screen and show a screen that was displayed just prior to the currently displayed screen. In other alternative embodiments, the input device includes one or more soft keys.

In preferred embodiments, a single keystroke is used to exit a Blank Screen and display at least one other screen. At least one of the at least one other screen is a Main Menu screen, an Express Bolus screen, an Easy Bolus screen, or a Status screen.

According to an embodiment of the invention, a method of programming an infusion device includes the steps of generating one or more menus, accessing the one or more menus, selecting a menu item from at least one of the one or more menus to access a set screen, modifying a control parameter displayed on the set screen, and either accepting the modification to the control parameter and exiting the set screen, or pressing the escape key to exit the set screen without accepting the modification to the control parameter. The infusion device includes a reservoir containing fluid for delivery into a user, a drive mechanism to force fluid from the reservoir, an input device that includes one or more keys including an escape key and accepts inputs from the user, a processor that uses control parameters to control the drive mechanism, and a display that receives information from the processor and visually displays screens containing the information for the user to see. The control parameters may be changed through inputs from the user.

According to another embodiment of the present invention, a programmable infusion device which includes a reservoir containing fluid for delivery into a user, a drive mechanism to force fluid from the reservoir, an input device that includes one or more keys and accepts inputs from the user, a processor that uses control parameters to control the drive mechanism, and a display that receives information from the processor and visually displays screens containing the information for the user to see. The control parameters may be changed through inputs from the user. The input device further includes generating means for generating one or more menus, accessing means for accessing one or more menus, selecting means for selecting a menu item from at least one of the one or more menus to access a set screen, modifying means for modifying a control parameter displayed on the set screen, accepting means for accepting the modification to the control parameter and exiting the set screen, and escape key means for to exiting the set screen without accepting the modification to the control parameter.

Other features and advantages of the invention will become apparent from the following detailed description, taken in conjunction with the accompanying drawings, which illustrate by way of example, various features of embodiments of the invention.

### BRIEF DESCRIPTION OF THE DRAWINGS

A detailed description of embodiments of the invention will be made with reference to the accompanying drawings, wherein like numerals designate corresponding parts in the several figures.

FIG. 1 is a simplified block diagram of an external infusion device and system in accordance with an embodiment of the present invention.

FIG. 2 is a perspective view of an external infusion device and system in accordance with an embodiment of the present invention.

FIG. 3 is a system diagram showing a computer in communication with a cradle, a perspective view of the cradle, and a top view of an external infusion device and an RF programmer in accordance with an embodiment of the present invention.

FIG. 4 is a top view of an RF programmer in accordance with an embodiment of the present invention.

FIG. 5 is a block diagram of an infusion device in communication with more than one RF programmer in accordance with an embodiment of the present invention.

FIG. 6 is a top view of a RF programmer with a display in accordance with another embodiment of the present invention.

FIG. 7 is a diagram of keystrokes to access screens from a Blank Screen in accordance with an embodiment of the present invention.

FIG. 8 is a diagram of keystrokes to access screens from a Main Menu in accordance with an embodiment of the present invention.

FIG. 9 is a diagram of keystrokes to access screens from a Bolus Menu in accordance with an embodiment of the present invention.

FIG. 10 is a diagram of keystrokes to access screens from a Set Bolus Menu in accordance with an embodiment of the present invention.

FIG. 11 is a diagram of keystrokes to enter and start an Easy Bolus in accordance with an embodiment of the present invention.

FIG. 12 is a diagram of keystrokes to enter and start an Express Bolus in accordance with an embodiment of the present invention.

FIG. 13 is a diagram of keystrokes to suspend fluid delivery in accordance with an embodiment of the present invention.

FIG. 14 is a diagram of keystrokes access screens from the Basal Menu in accordance with an embodiment of the present invention.

FIG. 15 is a diagram of keystrokes to create and start a Basal Profile in accordance with an embodiment of the present invention.

FIG. 16 is a diagram of keystrokes to access screens from the Prime Menu in accordance with an embodiment of the present invention.

FIG. 17(a) is a diagram of keystrokes to activate a Fixed Prime in accordance with an embodiment of the present invention.

FIG. 17(b) is a diagram of keystrokes to view a Prime History in accordance with an embodiment of the present invention.

FIG. 18 is a diagram of keystrokes to access screens from a Utilities Menu in accordance with an embodiment of the present invention.

FIG. 19 is a diagram of keystrokes to access screens from an Alarm Menu in accordance with an embodiment of the present invention.

FIG. 20 is a diagram of keystrokes to set the time and date.

FIG. 21 is a diagram of keystrokes to modify a list of RF devices that can communicate with the infusion device in accordance with an embodiment of the present invention.

FIG. 22 is a diagram of a Status Screen in accordance with an embodiment of the present invention.

FIG. 23 is a diagram of a display screen with a number to the right of a decimal point that is formatted differently than a number to the left of the decimal point, in accordance with an embodiment of the present invention.

FIG. 24(a) is a top view of an external infusion device that includes soft keys, displaying a Pattern Options screen, in accordance with an embodiment of the present invention.

FIG. 24(b) is a top view of an external infusion device that includes soft keys, displaying a Main Menu screen, in accordance with an embodiment of the present invention.

### DETAILED DESCRIPTION OF THE PREFERRED EMBODIMENTS

As shown in the drawings for purposes of illustration, the invention is embodied in an infusion device for infusing a liquid such as medication, chemicals, enzymes, antigens, hormones, sedatives, vitamins or the like, into a body of a user. The infusion device includes control parameters and data that are accessible by an individual through a menu structure. The menu structure provides a novel and unique framework to assist the individual to easily and efficiently locate the control parameters and/or data that are stored within the infusion device. The individual may manipulate some control parameters to change the performance of the infusion device. In preferred embodiments of the present invention, the infusion device is an external infusion pump, which is located outside of the body of a human user, for infusing insulin into the body of the user. In alternative embodiments, the infusion device is an internal infusion pump, which is implanted into the body of the user and uses an external programming device. In further alternative embodiments, the user is an animal. And in still further alternative embodiments; liquids other than insulin are infused into the body of the user.

Generally, preferred embodiments of the present invention have the capability to deliver insulin at a basal rate (continuous base rate of insulin measured in units/hour) and deliver a bolus (a measured number of units of insulin) to compensate for relatively sudden large increases in blood glucose, due to meals for example. In particular embodiments, the basal rate is programmable to deliver insulin at different rates throughout a day. Additionally, a temporary basal rate may be used to override the programmed basal rate or the basal delivery may be stopped and either manually restarted or programmed to start automatically. In other particular embodiments, the bolus is programmable to be delivered immediately as a single dose (normal), or to spread the dosage evenly over a defined period (square bolus), or a combination of a dosage to be delivered immediately and a dosage spread over a defined period (dual bolus). In alternative embodiments, the infusion device delivers other concentrations of insulin, or other liquids, and may use other limits on the delivery rate or bolus amount.

Individuals such as, health care professionals, infusion device users, and/or other individuals caring for users (such as trained relatives), may program the infusion device by accessing and changing various control parameters. The infusion device is manufactured with factory default values for the control parameters. A health care professional may modify one or more of the control parameters before issuing the infusion device to the user. Then the infusion device may be programmed by an individual as needed. It should be understood herein that the terms “program,” “programmed,” “programming,” and “programmable” are general terms that refer to a spectrum of operations, software manipulation, and data manipulation. Those terms are therefore not limited to creating, viewing, protecting, entering, deleting, or editing data, parameters, code, protocol, or the like. It should be noted that in general, the term “individual(s)” is used throughout this document to represent any person that might manipulate features of the infusion device 10, including the user that is receiving treatment from the infusion device 10. And in general, the term “user” is directed to, but not limited to, the entity receiving treatment from the infusion device 10.

### Hardware

As illustrated in FIGS. 1 & 2, preferred embodiments of the present invention include an infusion device 10 with a housing 12 that contains a processor 14 that sends information to, or receives information from, a memory 16, an LCD (Liquid Crystal Display) 18, a keypad 20, a power supply 22, a drive mechanism 24, a reservoir 26, a speaker 34, a vibrator 36, an IR (Infra-Red) transmitter/receiver 44, and an RF (Radio Frequency) transmitter/receiver 40. The infusion device 10 is of the type described in U.S. Pat. Nos. 4,685,903; 4,808,167; 4,850,972; 5,097,122; 5,080,653; 5,637,095; 5,665,065; and U.S. patent application Ser. No. 09/533,578, filed on Mar. 23, 2000, entitled “Cost-sensitive Application Infusion Device”; Ser. No. 09/429,352, filed on Oct. 28, 1999, entitled “Compact Pump Drive System”; and Ser. No. 09/334,858, filed on Jun. 16, 1999, entitled “External Infusion Device with Remote Programming, Bolus Estimator and/or Vibration Alarm Capabilities”; and PCT publications Serial No. US00/14954, filed on May 30, 2000, entitled “Cost-sensitive Application Infusion Device”; Serial No. US99/25414, filed on Oct. 28, 1999, entitled “Compact Pump Drive System”; and Serial No. US99/18977, filed on Aug. 17, 1999, entitled “External Infusion Device with Remote Programming, Bolus Estimator and/or Vibration Alarm Capabilities”; all of which are incorporated by reference herein. An individual navigates through the menu structure displayed on the LCD 18 by pressing a sequence of one or more keys (108, 110, 112, 114, and 116) on the keypad 20 to access and/or modify control parameters and data that have been stored in the memory 16 such as basal parameters, bolus parameters, priming parameters, alarms, limits, infusion set feedback, personal identification information, historical data (such as the times and volumes of the latest dosages, program changes, when priming occurred, and the like), power supply status, reservoir status, and the like. The processor 14 uses the control parameters to calculate and issue commands that affect the rate and/or frequency that the drive mechanism 24 forces fluid out of the reservoir 26, and into tubing 30 connected to an infusion set 32 that provides a fluid path into the user's body. The drive mechanism 24 includes a plunger slider (not shown) that is adapted to couple with a piston (not shown), which is part of the reservoir 26. The plunger slider moves the piston, which in turn forces fluid out of the reservoir 26.

The memory 16 stores programs, historical data, user defined information, and control parameters. In preferred embodiments, the memory is a Flash memory and SRAM; however, in alternative embodiments, the memory 16 may include other memory storage devices such as ROM, DRAM, RAM, EPROM, dynamic storage such as other flash memory, energy efficient hard-drive, or the like.

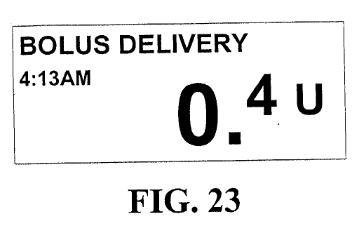

The LCD 18 displays menus, control parameters, options, operating modes, statuses, data, alarms, warnings, information, error messages, and the like. In preferred embodiments, the LCD 18 has sufficiently fine resolution to display words and numbers and to show graphics such as a meter bar or a sliding scale to indicate, for example, the amount of power remaining in the power supply, or the amount of medicament remaining in the reservoir, how far an individual has scrolled through a list of data, and the like. Critical information is shown in larger font sizes than less important information. In particular embodiments, decimal numeric values appear on the LCD 18 with the values on one side of a decimal point having a different font, such as a different size, style, color, spacing, super scripted, subscripted, underlined, bolded, italicized, or the like compared to the numeric values that appear on the other side of the decimal point. As an example, a value after a decimal point may be both a smaller font and superscripted compared to a value before a decimal point, as shown in FIG. 23. In alternative embodiments, many font sizes are used with the most important information being displayed with the largest font sizes and the least important information shown with the smallest font sizes. In other alternative embodiments, the same or similar font sizes are used for all information.

Preferably, the LCD 18 has a backlight that the individual may activate to illuminate the LCD 18 as needed. In alternative embodiments, the LCD 18 may be replaced with an LED (light emitting diode) display, plasma screen, a touch screen, a color LCD, or the like. And the display resolution may be increased to display icons to represent data, control parameters, function keys, and the like. In other alternative embodiments, the display is eliminated from the infusion device and feedback is provided to the individual through sound, vibration, brail, or visually displayed on another device that has received information from the infusion device.

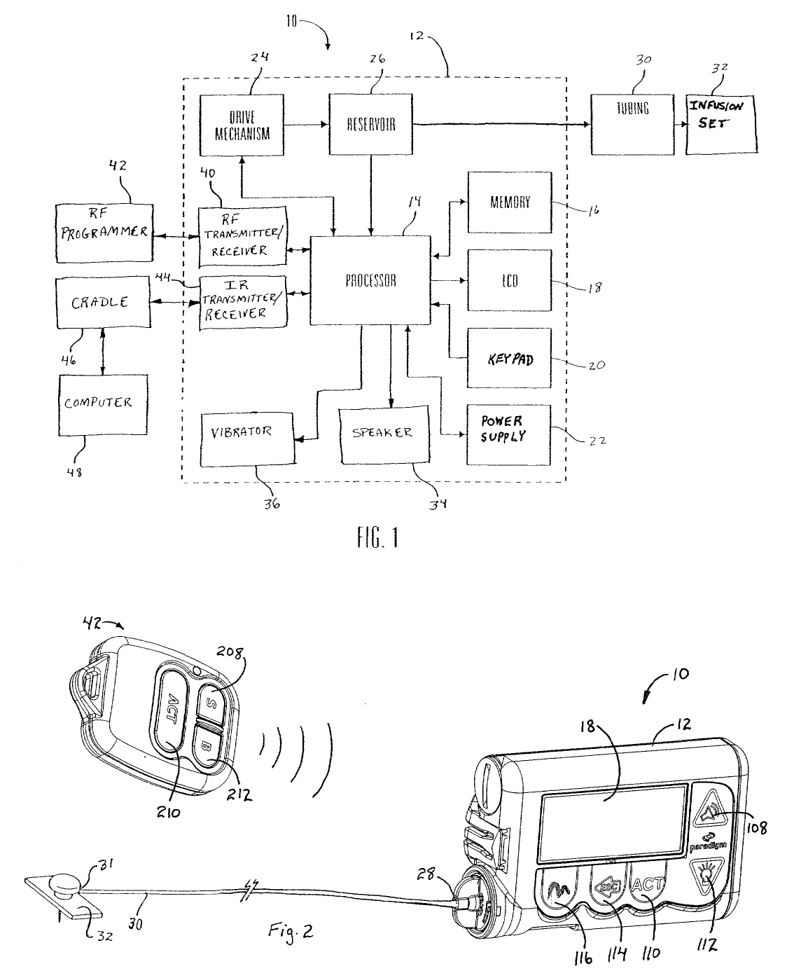

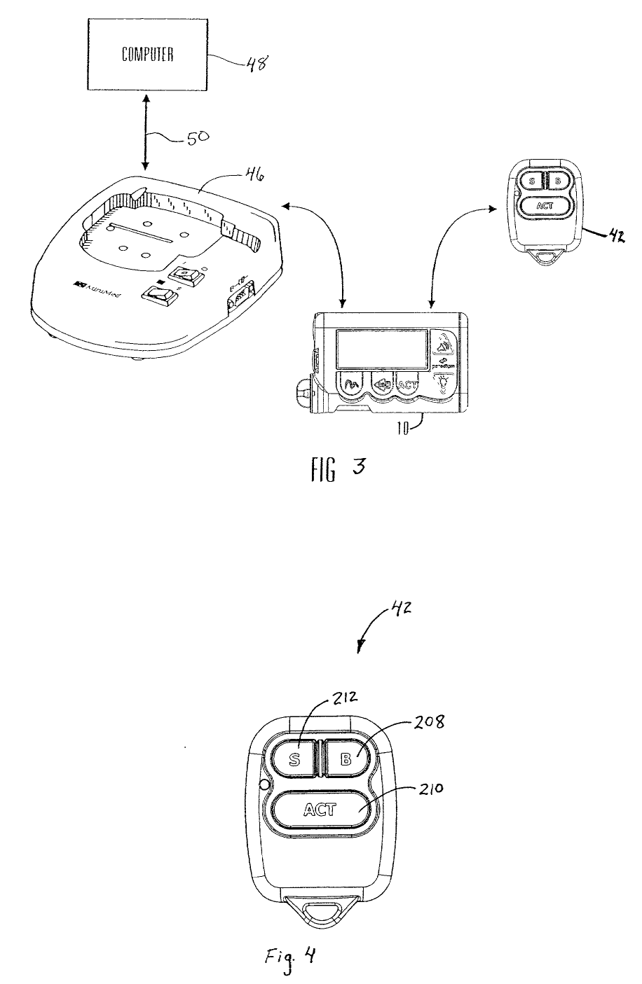

The keypad 20 of a preferred embodiment, shown in FIG. 2, has five keys including an Up-Arrow key 108, an ACT (activate) key 110, a Down-Arrow key 112, an Esc (escape) Key 114, and an Express Bolus key 116. The keypad 20 provides the primary means for the individual to provide input to the infusion device 10. The individual presses keys on the keypad 20 to display and scroll through information, call up menus, select menu items, select control parameters, change control parameters (change values or settings), enter information, turn on the backlight, and the like. In alternative embodiments, the keypad 20 may utilize more or less keys or have different key arrangements than those illustrated in the figures.

In preferred embodiments, the keys 108, 110, 112, 114, and 116 are membrane switches with metal domes, which are known for reliability, durability, and low profile. In alternative embodiments, other types of keys such as a rubber key pad, diaphragm covered switches, or the like, or other input interfaces such as buttons, a keyboard, mouse, joystick, voice activated controller, a touch screen, or the like may be used. In further alternative embodiments, the keypad 20 may be omitted and an LCD may be used as a touch screen input device or devices other than the infusion device 10, such as an RF programmer 42, a computer 48 connected to a cradle 46, a PDA (Personal Digital Assistant), a phone, or the like may be used to provide an interface between the individual and the infusion device 10.

The power supply 22 of a preferred embodiment provides the power to operate the infusion device 10, and in preferred embodiments, the power supply is at least one battery. In particular embodiments, the power supply is one or more replaceable AAA batteries. Energy storage devices such as capacitors, backup batteries, or the like provide temporary power to maintain the memory during power supply replacement. In alternative embodiments, the power supply is one or more button batteries, zinc air batteries, alkaline batteries, lithium batteries, lithium silver oxide batteries, AA batteries, or the like. In still further alternative embodiments, the power supply is rechargeable.

The speaker 34 and/or the vibrator 36, which provide feedback to the individual from the infusion device 10, are activated or deactivated by the individual by accessing control parameters through the menu structure in preferred embodiments. Feedback may include signals that notify the individual of modifications to the control parameters, announce that the infusion device 10 is about to initiate a particular operation, indicate a mode of operation, provide a warning (for instance to indicate a low fluid level in the reservoir or low battery power), present an alarm (such as from a timer or a clock), present an error message to indicate a malfunction of the system (such as an occlusion that restricts the delivery of the fluid, a software error, or the like), request input, confirm that communication has been established, and the like. Alarms and warnings may start out at a low level and escalate until acknowledged by the user. In preferred embodiments, the alarm intensity changes over time. If the individual does not respond to the alarm, the alarm may change tone, change volume, increase the vibration amplitude or frequency, project a brighter light or a different color light, flash, flash at a different frequency, and the like. In alternative embodiments, the intensity may vary up or down. In other alternative embodiments, the intensity is constant. In further alternative embodiments, the intensity changes by activating different alarm types over time.

In further embodiments, both an audible alarm and a vibration alarm may be given at the same time or alternately pulsed. The non-visual feedback provided by the speaker 34 and/or the vibrator 36 is especially beneficial to visually impaired users. In other embodiments, other ways are used to provide feedback to the individual such as lights, LEDs, LCD messages, a transmitted message, Braille, electrical scintillation, voice messages, and the like.

In preferred embodiments, the infusion device 10 can send or receive information through the IR transmitter/receiver 44. Information, control parameters, programs, and the like may be transmitted to other devices, and/or the infusion device 10 may receive communications from other devices to store in the memory 16 or for the processor 14 to use to control the drive mechanism 24. For example, a health care professional can use a computer 48 to configure the infusion device 10 so that it provides or restricts access to certain control parameters. In other examples, data generated at the infusion device 10 can be used alone or combined with data from a glucose meter, a glucose monitor, a glucose sensor, and/or other devices (all of which are not shown) to assist the user and/or the health care professional in making intelligent therapy decisions. Moreover, the information, programs, and data may be downloaded to a remote or local PC, laptop, or the like, for analysis and review by a MiniMed employee or a trained health care professional.

In particular embodiments, the data may be downloaded through a cradle 46. For example, the cradle 46 may be used to connect to a remotely located computer 48 such as a PC, laptop, or the like, over communication lines 50, by modem or wireless connection, as shown in FIG. 3. In preferred embodiments, the cradle 46 establishes communication with the infusion device 10 and data is transferred between the computer 48 and the infusion device 10, as described in U.S. Pat. No. 5,376,070, entitled “Data Transfer System For An Infusion Pump”; U.S. patent application Ser. No. 09/409,014, filed on Sep. 29, 1999, entitled “Communication Station and Software For Interfacing With An Infusion Pump, Analyte Monitor, Analyte Meter or the Like”; and U.S. Design patent application Ser. No. 29/087,251, filed on Apr. 29, 1998, entitled “Communication Station For An Infusion Pump”; and Ser. No. 29/131,830, filed on Oct. 31, 2000, entitled “Communication Station For An Infusion Pump and Monitor”; and PCT Patent Application Serial No. US99/22993, filed on Sep. 30, 1999, entitled “Communication Station and Software for Interfacing with an Infusion Pump, Analyte Monitor, Analyte Meter or the Like,” all of which are incorporated by reference herein. In alternative embodiments, the cradle establishes communication with the infusion device using RF, optical, hardwire contacts, or the like. In preferred embodiments, the cradle 46 establishes IR communication with the infusion device 10. In alternative embodiments, the cradle establishes communication with the infusion device using other media such as RF signals, direct electrical contacts, laser, light frequencies other than IR, sound waves, ultra sonic waves, or the like.

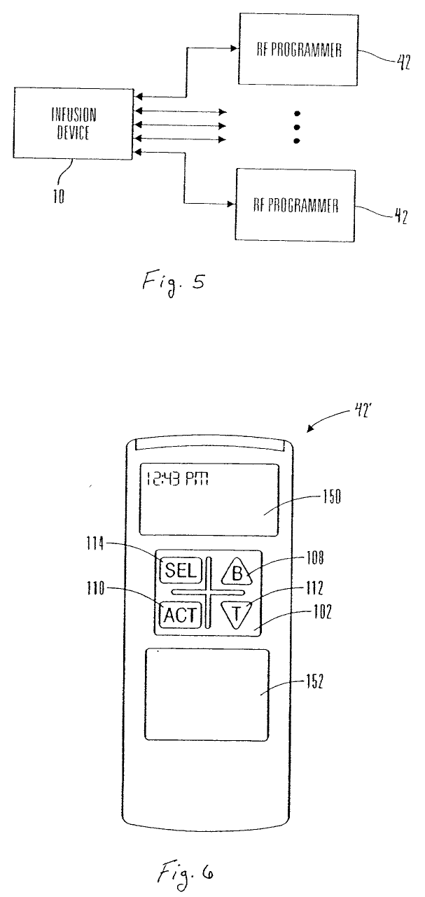

In preferred embodiments, the RF programmer 42 is optional equipment that may be used to communicate with the infusion pump 10, as shown in FIGS. 1-5, and described in U.S. patent application Ser. No. 09/334,858, filed on Jun. 16, 1999, entitled “External Infusion Device with Remote Programming, Bolus Estimator and/or Vibration Alarm Capabilities”; and PCT publication Serial No. US99/18977, filed on Aug. 17, 1999, entitled “External Infusion Device with Remote Programming, Bolus Estimator and/or Vibration Alarm Capabilities”; which are incorporated by reference herein. In alternative embodiments, the RF programmer is required. In preferred embodiments the user may modify control parameters in the infusion device 10 so that more than one RF programmer 42 may be used to communicate with the infusion device 10, as shown in FIG. 5. In preferred embodiments, the RF programmer 42 is used to establish communication with the infusion device 10 and then to enter and start an “easy bolus” delivery of fluid (described later) or suspend fluid delivery. In alternative embodiments, the RF programmer is used to access data and/or modify one or more control parameters, such as a bolus amount, a bolus profile, a bolus time, basal rates, priming functions (perhaps including rewinding the plunger slider), self tests, setting date and time, reviewing stats, and the like. In further alternative embodiments, an infusion device and one or more RF programmers are paired at the factory, or in the doctors office, and may not be changed by an individual.

The remote programming capability of the RF programmer 42 combined with audio and/or vibratory feedback from the infusion device 10 allows individuals to readily access the most commonly used operations of the external infusion device 10 without having to touch the infusion device 10 or see the LCD 18. This is especially beneficial to users that prefer to carry the infusion device 10 discreetly, such as under clothing, since they do not have to handle the infusion device 10 to issue program changes and receive feedback. In preferred embodiments, the infusion device 10 confirms receipt of instructions from the RF programmer 42 by issuing one or more audible beeps or tactile vibrations. In alternative embodiments, the RF programmer 42 includes a receiver. Additionally, it may provide a feedback signal such as a sound or vibration to indicate that the commands have been received and acknowledged by the infusion device 10.

In preferred embodiments, the RF programmer 42 has three keys including an S key 208, an ACT (activate) key 210, and a B key 212, as shown in FIGS. 2-4. In alternative embodiments, the RF programmer may have a greater or smaller number of keys depending on the type of information that is to be exchanged with the infusion device 10. In further alternative embodiments, other devices may communicate with an infusion device such as an RF programmer 42′ with a display 150 and/or a keypad 152, such as shown in FIG. 6. In still further alternative embodiments, the keypad 20, LCD 18, speaker 34, vibrator 36, and/or the IR transmitter/receiver 44 are omitted from the infusion device, and all modifications to programming and all data transfer is handled through an RF programmer. For instance, since the RF programmer 42′ includes a display 150, it may use a programming protocol employing the same key sequences as those described for using the keypad 20 to program the infusion device 10. In particular alternative embodiments, the RF programmer 42′ receives signals back from the infusion device echoing commands or indicating receipt of commands. The RF programmer 42′ may indicate receipt of a response from the infusion device by displaying information of the display 150 or by a speaker, vibrator, or the like. This is especially beneficial for use with an internal infusion device. In further alternative embodiments, the RF programmer 42′ may use a more sophisticated programming technique, such as single key programming, if the display 150 includes the capability to use touch screen techniques, or may use additional keys in the keypad 152 that are specifically identified with particular programming features on the infusion device 10. In other alternative embodiments, the keypad 20, LCD 18, speaker 34, vibrator 36, and/or the IR transmitter/receiver 44 are duplicated in both the infusion device and the RF programmer. The individual can receive feedback from the infusion device even if most or all of the programming is conducted with the RF programmer, or the individual can enter and retrieve data through the RF programmer or the infusion device directly. In still other alternative embodiments, other devices may communicate with the infusion device such as blood glucose monitors, blood glucose meters, or the like.

### Software

Individuals such as, health care professionals, infusion device users, and/or other individuals caring for users (such as trained relatives), may program the infusion device 10 by accessing and changing various control parameters. In embodiments of the invention, many of the programming features are organized under a menu structure to help individuals locate the information they wish to view and the control parameters they wish to view or adjust.

In preferred embodiments, a user may view data and access several control parameters through the menu structure of the infusion device 10. Through the menu structure, the user can select and customize at least two different basal patterns, and/or a temporary basal rate, program and activate at least two types of boluses, suspend fluid delivery, set the time of day and calendar date, set maximum allowable values for basal rates and bolus amounts, choose a language for the display, activate blocking, define which if any RF devices will communicate with the infusion device 10, review historical logs and statistics and settings, select a therapy (such as an insulin formulation concentration, medication, sedative, hormone, vitamins, or the like), reset control parameters to their factory default values, reset control parameters to the values set by a health care professional, command the infusion device 10 to rewind the plunger slider (usually so that a replacement reservoir can be installed), command the plunger slider to engage the piston of the reservoir 26, and prime the tubing 30 and infusion set 32.

It should be understood herein that the term “screen”, used alone or with a modifier such as “information screen,” “select screen”, “set screen,” “display screen,” “Basal Menu screen,” or the like refers to a set of indicia displayed for the individual to observe. The term “display,” when used as a noun, generally refers to the hardware device employed to show the screen. As described above, in preferred embodiments the screens are shown on the LCD 18, but in other embodiments the screens may be generated by a LED display, a touch screen display, a computer monitor, a PDA display, a phone display, a Braille device, a voice synthesizer, or the like. In further alternative embodiments, more than one component may be used to display different screens. In general, each of the display screens falls into one of four categories: information, select, set, or confirmation.

Information screens display information such as statuses, statistics, alarm messages, error messages, warnings, and historical data. More specifically, the information screens might display the dates and times that errors occurred, alarms were activated, priming was commanded, basal rates were modified, boluses were delivered and their amounts, and the like. When an information screen is displayed, the Up-Arrow and Down-Arrow keys (108, 112) are used to scroll through the information. The ACT key 110 and/or Esc key 114 are used to enter and/or exit the information screen.

Select screens display items for the user to select such as menu items, ‘yes’ or ‘no’, ‘on’ or ‘off’, basal pattern ‘standard’, ‘A’, or ‘B’, and the like. When a select screen is displayed, the Up-Arrow and Down-Arrow keys (108, 112) are used to highlight a selection. Then the ACT key 110 is used to select the highlighted selection, which enters the selection and exits the select screen. Or the Esc key 114 is used to exit the select screen without entering the selection.

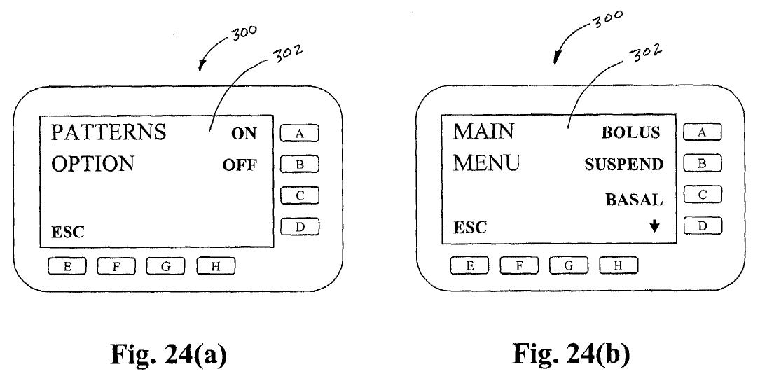

In alternative embodiments, “soft keys” are used. “Soft keys” refers to keys that perform a function that is described by a label on a display. As the labels on the display change, the functions of the keys change. The title of the screen and the items that may be selected are shown on the display. In particular alternative embodiments, each item that may be selected from a select screen is displayed adjacent to a key. An item is selected by pressing the key adjacent to the item. For example, referring to FIG. 24(a), when a Patterns Option screen is shown on a display 302 of an external infusion device 300, key ‘A’ would be pressed to enter ‘On’, key ‘B’ would be pressed to enter ‘Off’, and key ‘E’ would be pressed to escape from the Pattern Options screen. Keys ‘C’, ‘D’, ‘F’, ‘G’, and ‘H’ would have no effect while the Patterns Option screen is displayed. Continuing the example, the same display 302 of the same external infusion device 300 might show a Main Menu screen as shown in FIG. 24(b), and key ‘A’ would be pressed to select ‘Bolus’, key ‘B’ would be pressed to select ‘Suspend’, key ‘C’ would be pressed to select ‘Basal’, key ‘D’ would be pressed to select to scroll down, and key ‘E’ would be pressed to escape from the Main Menu screen. Keys ‘F’, ‘G’, and ‘H’ would have no effect while the Main Menu is displayed. In further alternative embodiments, more or less keys may be used, and different labels or no labels may be used on the keys. In other alternative embodiments, instead of listing items next to the keys, the display may show each item next to a symbol, number, or letter that represents the key used to select that item. In other alternative embodiments, “soft keys” may be used along with dedicated keys. For example, up and down arrow keys may be used to scroll through a list to display different portions of the information while “soft keys” are used to select an item from the list. Other dedicated keys that may be available include, but are not limited to, an Esc key, a light key, a suspend key, a basal key, a bolus key, a prime key, a utilities key, and a status key, while other keys function as soft keys.

Set screens display input prompts for the user to enter modifications to control parameters or information. When a set screen is displayed a value on the display flashes on and off prompting the user to change or confirm the flashing value. Control parameters that might be changed in a set screen include a bolus duration, a bolus amount, a basal start time, a basal amount, an off duration, the hour of the day, the minute of the hour, the day of the month, the month of the year, the year, an RF programmer ID number, and the like. When a set screen is displayed, the Up-Arrow and Down-Arrow keys (108, 112) are used to increment or decrement the flashing value. Then the ACT key 110 is used to enter the flashing value and exit the set screen, or the Esc key 114 is used to exit the set screen without entering the flashing value. In alternative embodiments, other type of keys such as number keys, left and right arrow keys, letter keys, keys with symbols, or the like may be used to modify control parameters. In other alternative embodiments, methods other than flashing are used to prompt the user to change or confirm a value, such as underlining, highlighting, a color change, a tone change, animation, and the like.

In preferred embodiments, critical select screens and set screens are followed by a confirmation screen. The confirmation screen shows an item or value that has been selected or set in a previous screen, and requires that the individual confirm the information shown. Typically, confirmation screens are a subset of select screens. In particular embodiments, the individual selects ‘Yes’ to confirm and accept the displayed item or value, and selects ‘No’ to discard the item or value. Preferably, if the item or value is discarded, the display returns to a previous screen to allow the individual to select a different item or enter a different value. In preferred embodiments, the default on a confirmation screen is ‘No’. This forces the individual to take a definite action of scrolling to ‘Yes’ and pressing the ACT key 110 to confirm the item or value rather than simply pressing the ACT key 110 an extra time. Pressing the ACT key 110 alone several times would not be sufficient to confirm a critical value. As an example, a confirmation screen is used to verify the insulin type before priming, as shown in FIG. 16 and discussed in detail later under PRIMING. In alternative embodiments, other defaults are used. In other alternative embodiments, no confirmation is used.

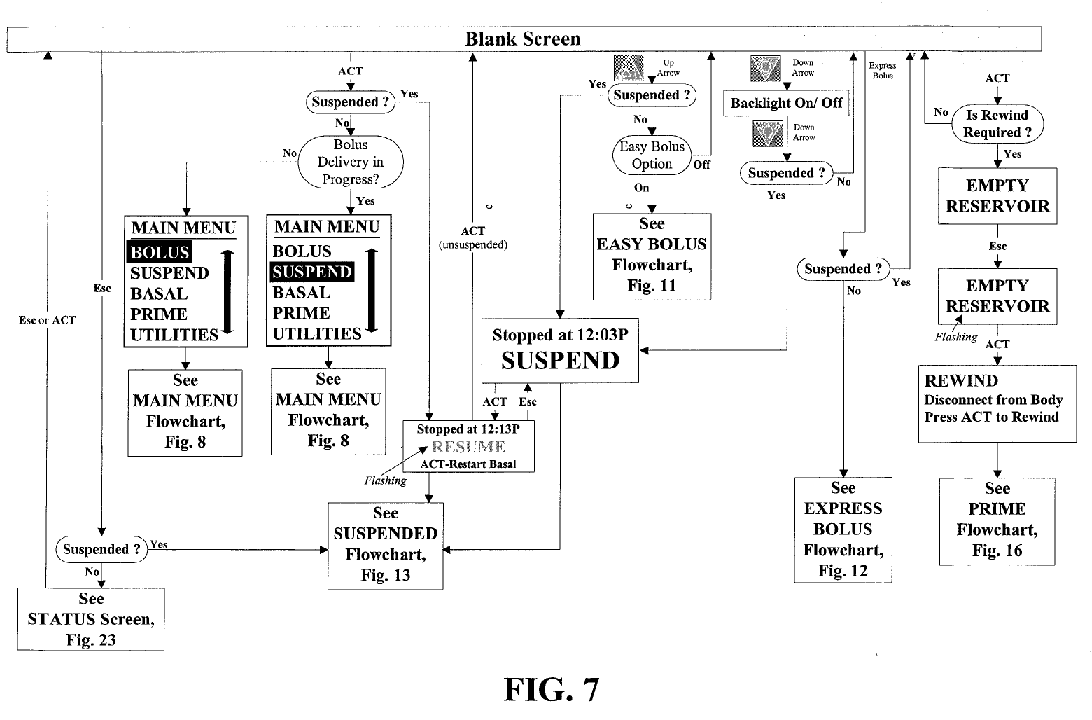

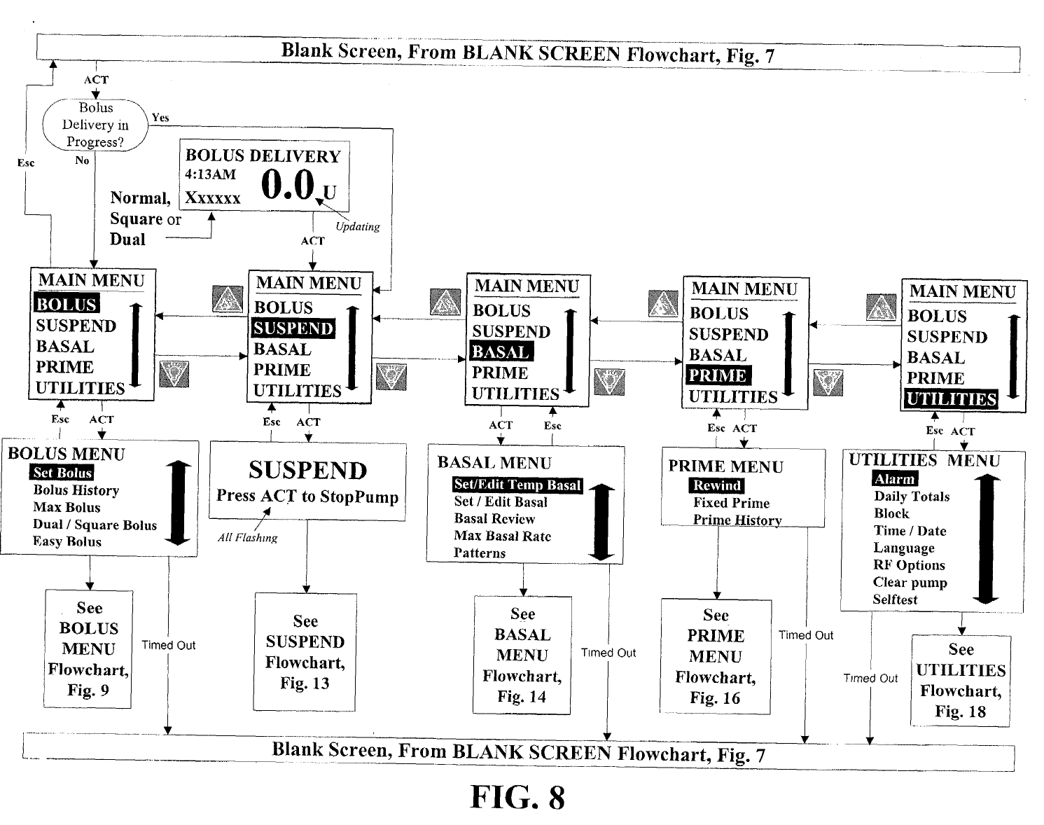

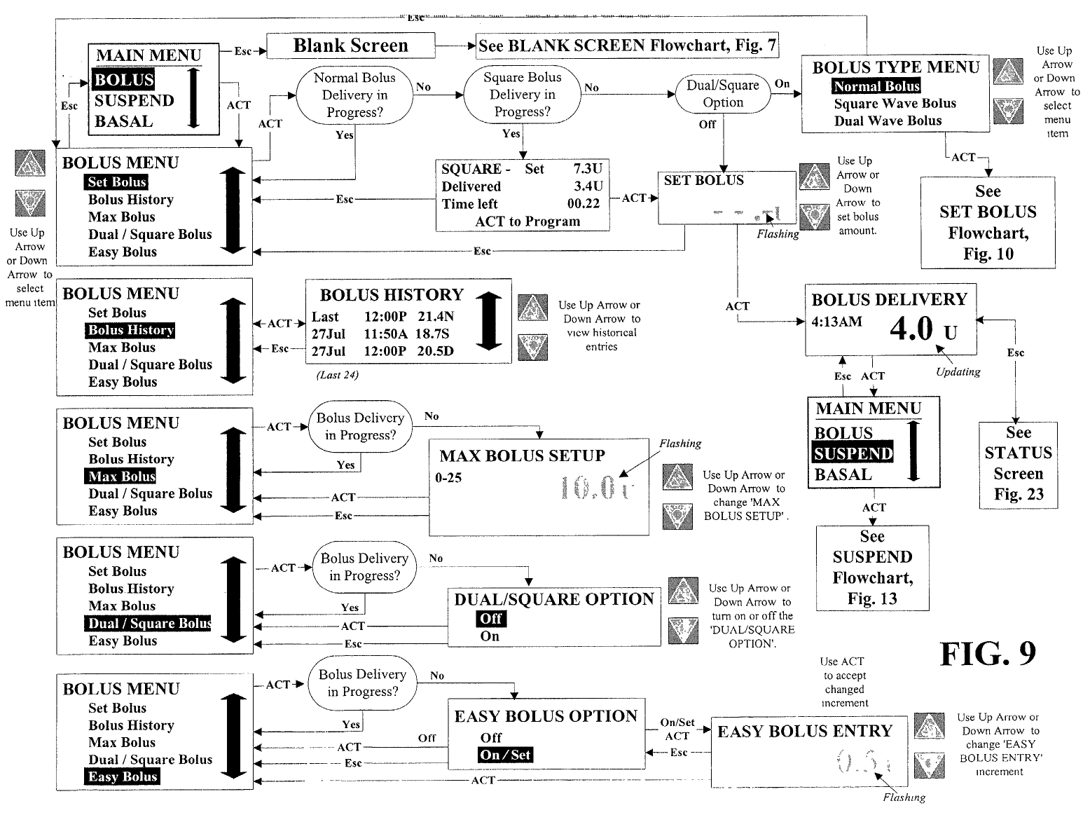

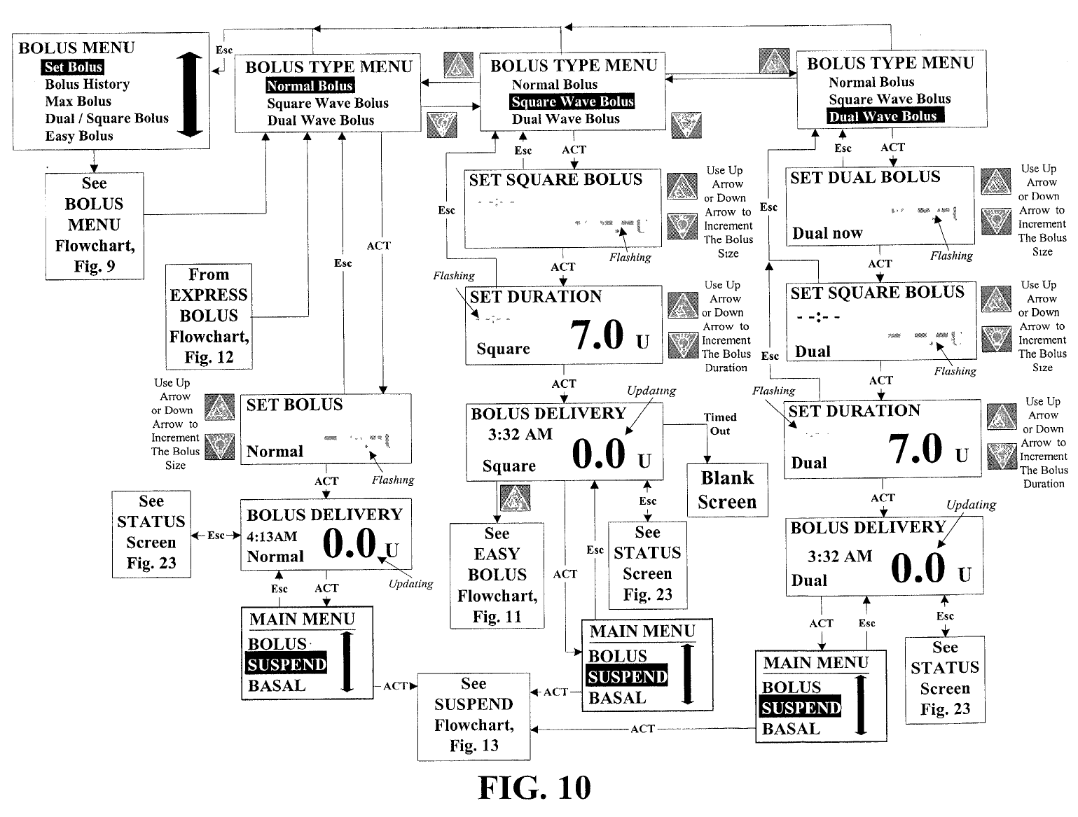

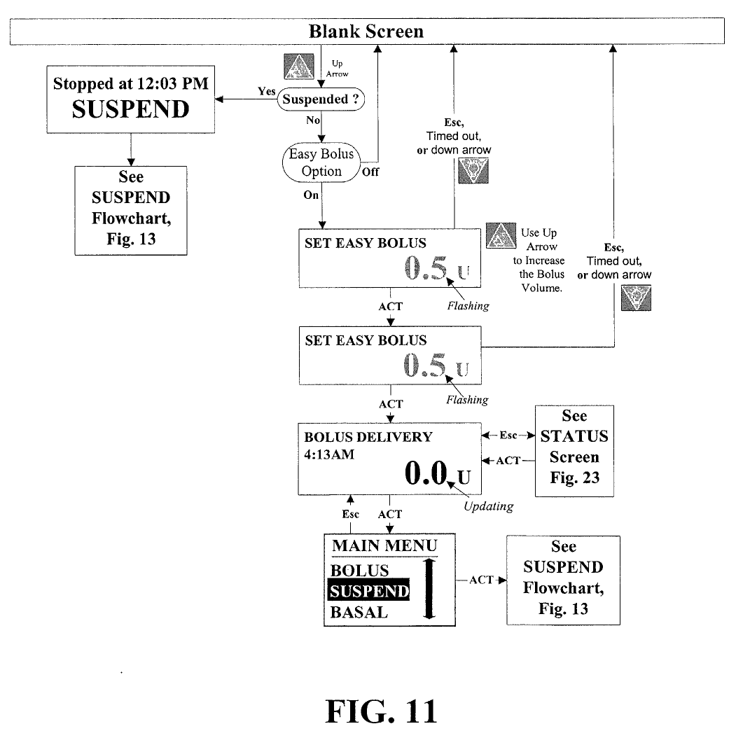

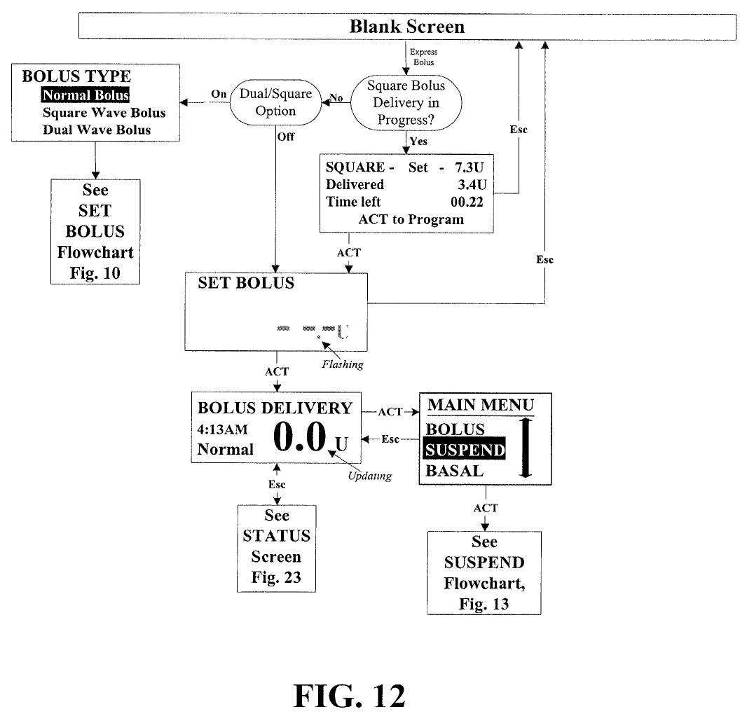

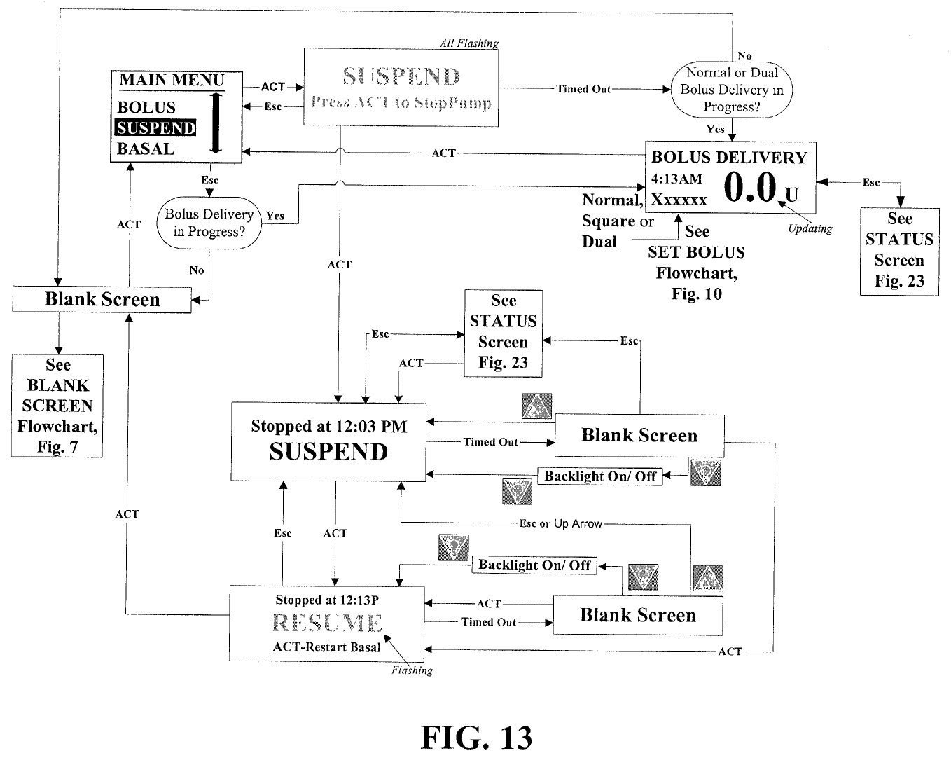

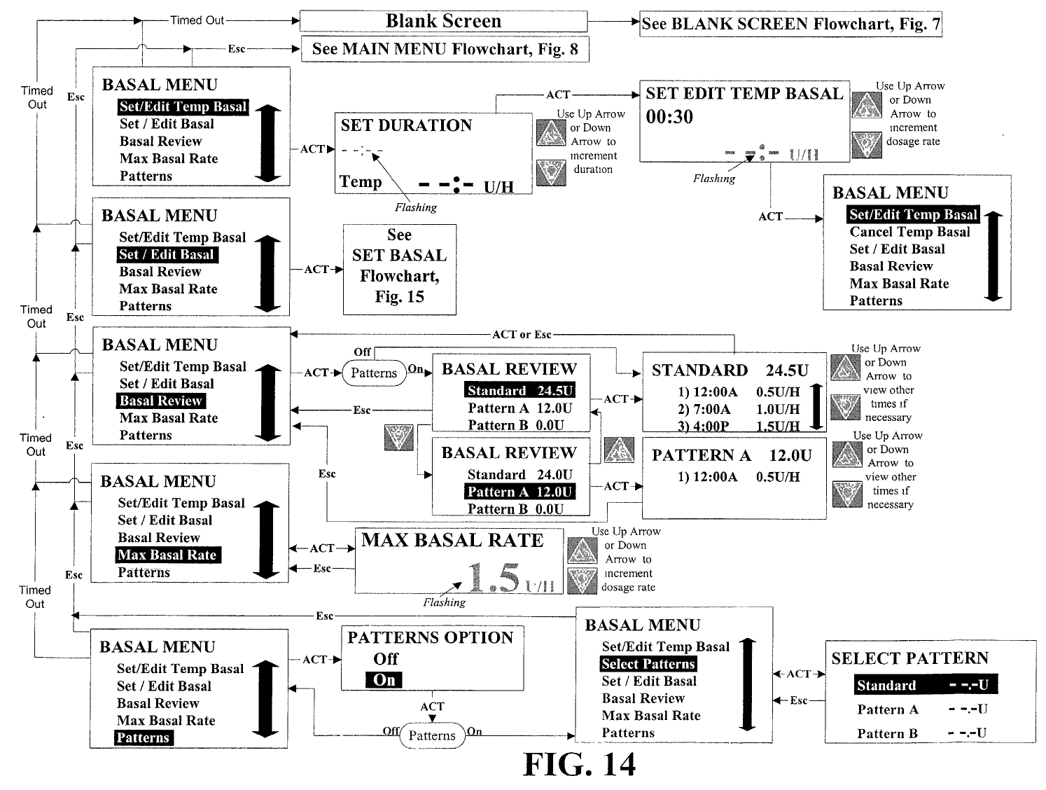

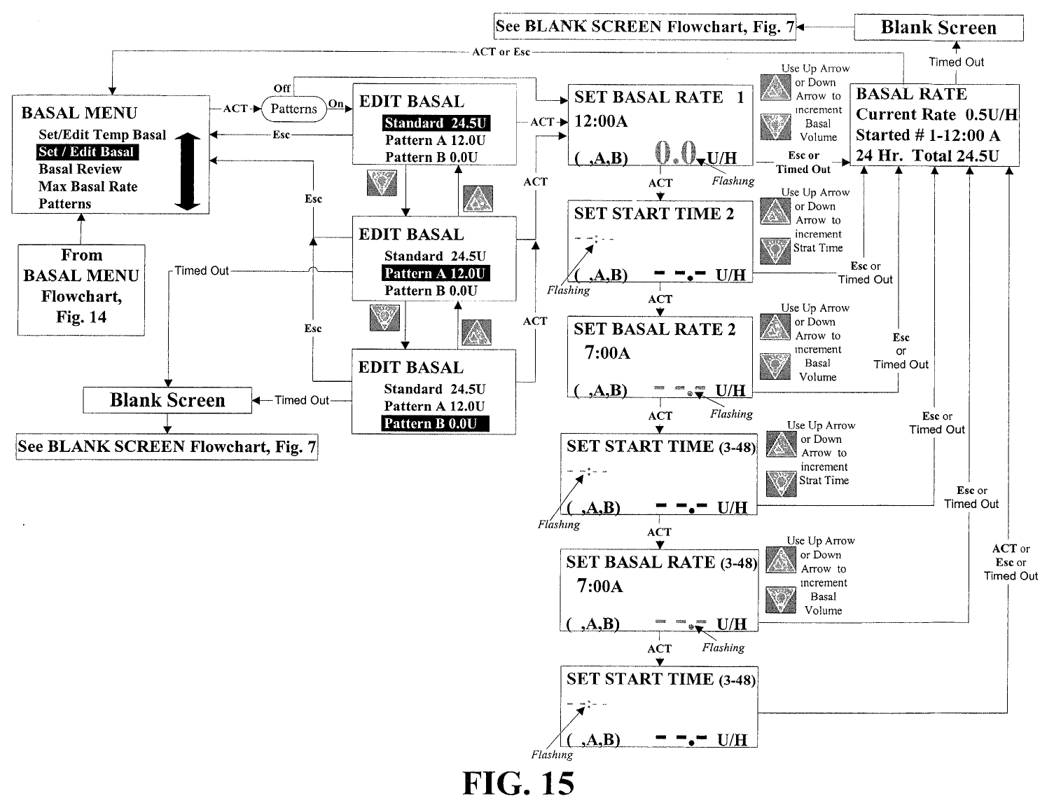

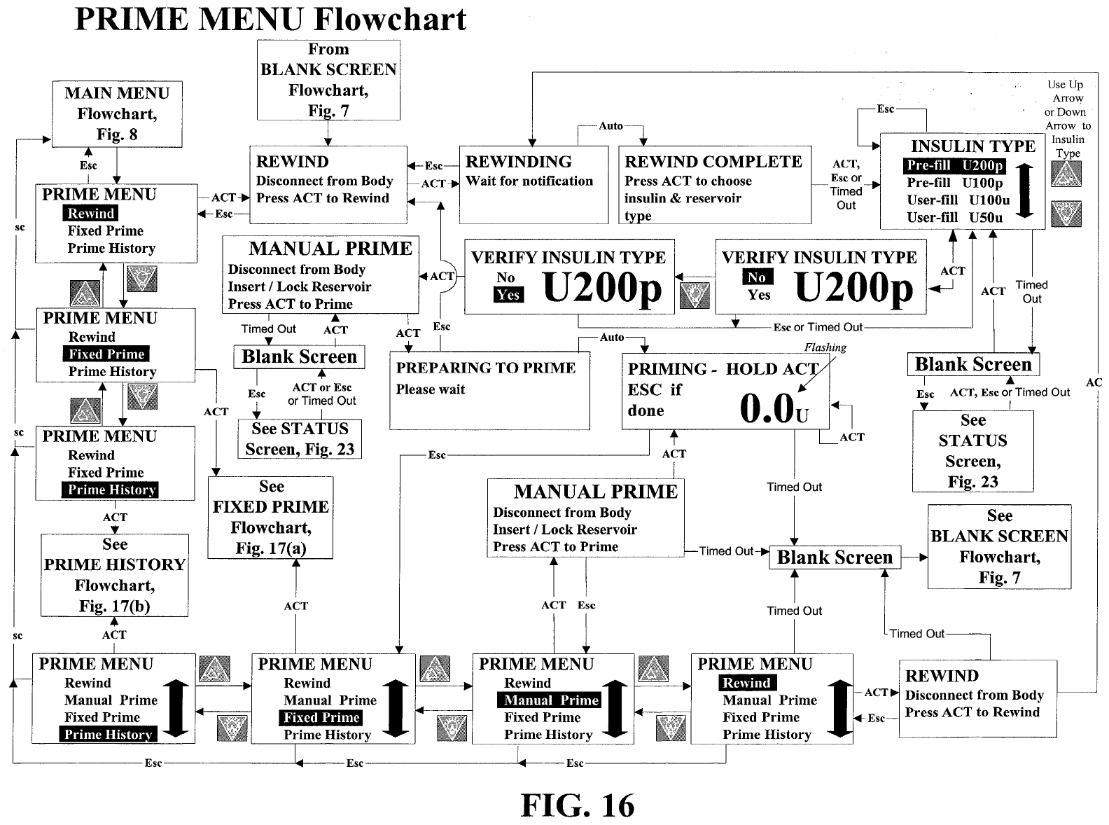

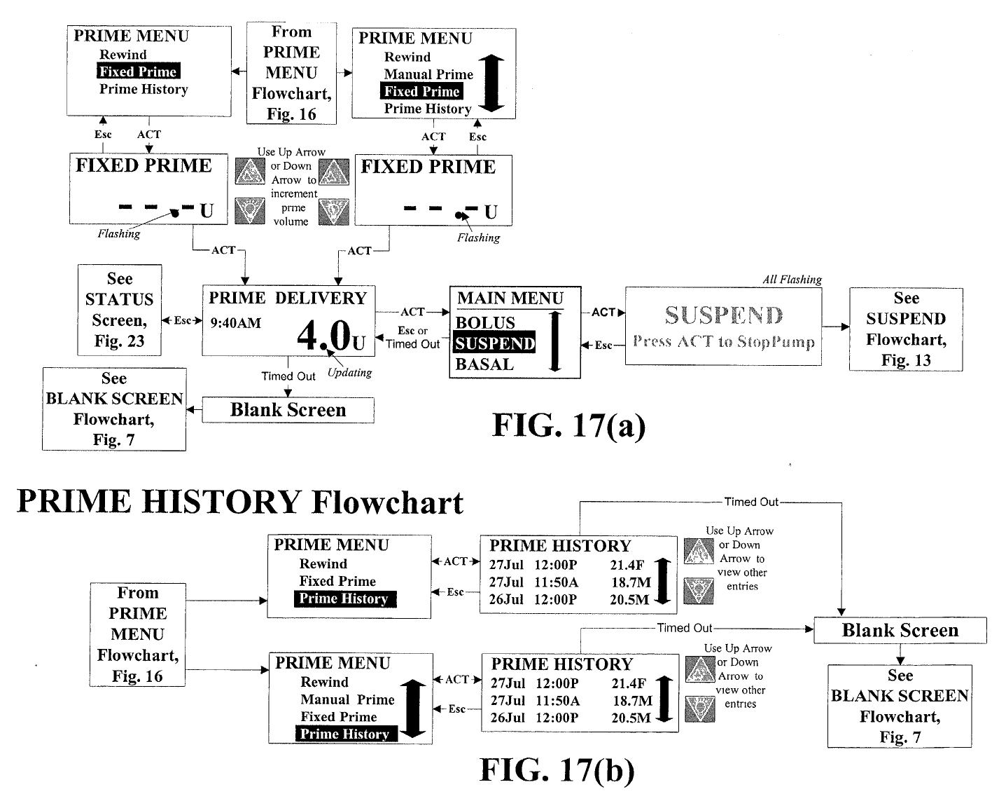

### Blank Screen (FIG. 7)

In preferred embodiments, the infusion device 10 includes energy management software that turns off the LCD backlight and/or changes the display to a Blank Screen after a Time-Out delay has expired (measured since the last key operation). Generally, when a screen has timed-out, the display returns to the Blank Screen, as shown in FIGS. 7-22. This is also considered a safety feature, because if a user becomes confused and does not know how to exit a screen then the user can wait for the infusion device 10 to time-out, and the display will return to the Blank Screen. In this way, the Blank Screen is the ‘Home’ screen. Preferably, the Time-Out delay is a different duration depending on the screen that is displayed. For example, if the Blank Screen is displayed, the duration that the LCD backlight remains lit after a keystroke is shorter than the duration it would remain lit if the screen were displaying information. In alternative embodiments, the Time-Out delay is the same for all screens. In other alternative embodiments, the backlight and/or the display are only on while a key is held down.

Most of the time, the Blank Screen is displayed. In preferred embodiments, the Blank Screen is not entirely blank. It continuously displays at least one pixel such as a header, boarder, an Icon, a moving shape, a date, a time of day, an animation, or the like as an indication to the user that the infusion device is powered and operational. In preferred embodiments, the Blank Screen includes other indicia, symbols, icons, pixels, or the like to provide warnings, indicate a mode of operation, indicate that interaction is required from an individual, or the like. For example, in particular embodiments, open circles displayed on the Blank Screen indicate that a basal or bolus delivery is in process, and closed (solid) circles indicate that an alarm has been triggered requiring interaction with the individual. In other particular embodiments, icons of empty or partially filled containers indicate that the reservoir is near empty or that the battery voltage is low. In alternative embodiments, a dedicated pixel, icon, or symbol is used for each item to be communicated. For example, one symbol would be displayed to indicate that a basal profile is active and a different symbol would indicate that a bolus is being delivered. Many symbols might be displayed simultaneously to communicate many aspects about the status of the infusion device.

In preferred embodiments, the individual can exit the Blank Screen and go to at least one other screen with a single keystroke. In alternative embodiments, more than one keystroke is required to exit the Blank Screen.

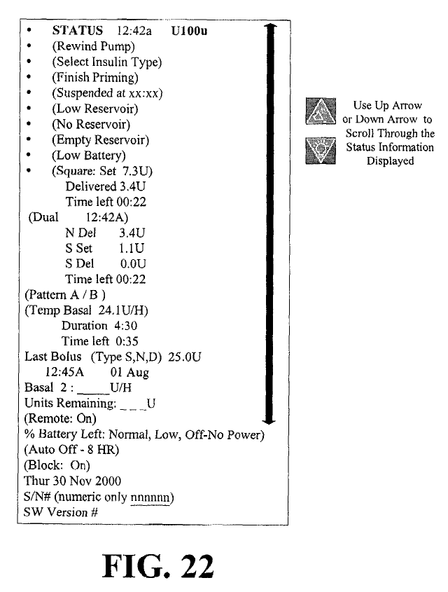

In particular embodiments, the individual may access up to four different screens from the Blank Screen when the pump is not suspended, including a Main Menu screen (shown in FIG. 8) by pressing the ACT key 110, a Set Easy Bolus screen (shown in FIG. 11) by pressing the Up-Arrow key 108, a Set Bolus screen (shown in FIG. 12) by pressing the Express Bolus key 116 or a Status screen (shown in FIG. 22) by pressing the Esc key 114, all of which are shown in FIG. 7. When the Blank Screen is displayed, the Down-Arrow key 112 operates the LCD backlight. If the infusion device 10 is suspended and the Blank Screen is displayed, then pressing the ACT key 110, the Up-Arrow key 108, or the Esc key 114 will take the user to a Suspend screen, as shown in FIG. 7. However, pressing the Express Bolus key 116 does nothing, and pressing the Down-Arrow key 112 once turns on the backlight and a second press displays a Suspend screen, as shown in FIG. 7.

Furthermore, while in any screen including the Blank screen, if the reservoir 26 is empty, pressing the ACT key 110 causes the display to automatically show a Warning screen, informing the user that the reservoir is empty. Before pressing the ACT key 110, all of the other keys function as though the reservoir were not empty until the ACT key 110 is pressed. To exit the Warning screen, an individual may press the Esc key 114, which causes the warning message to flash. Then when the ACT key 110 is pressed again, a rewind screen is displayed as if the user had selected “Rewind” from a Prime Menu, as shown in FIG. 16. In alternative embodiments, a rewind screen is automatically displayed when the reservoir is empty.

In alternative embodiments, more or less screens may be accessed by pressing a single key while the Blank Screen is displayed.

In particular embodiments, a Warning screen includes full circles. In other embodiments, warnings are displayed on Warning screens as symbols, messages, color changes, flashing, a special font style, or the like. Warnings may include low battery voltage, empty and/or low reservoir, excessive bolus requested (a normal bolus amount but more frequent than usual), unusually large bolus requested, unusually low total fluid used for the day, and the like.

### Main Menu (FIG. 8)

In preferred embodiments, most of the control parameters are accessed through the Main Menu. Menu items contained within the Main Menu include: Bolus, Suspend, Basal, Prime, and Utilities, as shown in FIG. 8. In alternative embodiments, more, less, or different menu items are contained within the Main Menu. Generally, when the Main Menu is displayed, the top menu item is highlighted by default. However, in particular embodiments, other menu items may be highlighted automatically upon entering the Main Menu, especially when the probability of selecting a particular menu item is higher due to a function that the infusion device 10 is currently performing. For example, generally the ‘Bolus’ menu item (the first menu item) is highlighted when the main menu is displayed because there is a higher probability that an individual will need to modify a bolus parameter than make any other modifications to control parameters while the infusion device is in use. But, when the infusion device 10 is already delivering a bolus, the ‘Suspend’ menu item (the second item on the Main Menu) is highlighted by default when the Main Menu is displayed, since there is a higher probability that the individual will select the ‘Suspend’ menu item over other menu items while a bolus is being delivered.

The Down-Arrow key 112 and the Up-Arrow key 108 are used to highlight other menu items. Only one menu item is highlighted at a time. In preferred embodiments, the menu items are wrapped so that pressing the Up-Arrow key 108 when the top menu item is already highlighted causes the bottom menu item to be highlighted, and pressing the Down-Arrow key 112 when the bottom menu item is already highlighted causes the top menu item to become highlighted. In alternative embodiments, the menu items are not wrapped so that pressing the Up-Arrow key 108 when the top menu item is already highlighted has no effect, and pressing the Down-Arrow key 112 when the bottom menu item is already highlighted, also has no effect. The Act key 110 is used to select the highlighted menu item.

### Bolus (FIGS. 9-12)

In preferred embodiments, the Bolus Menu is displayed by selecting the Bolus menu item from the Main Menu, as shown in FIG. 8. The menu items within the Bolus Menu include: Set Bolus, Bolus History, Max Bolus, Dual/Square Bolus, and Easy Bolus, as shown in FIG. 9.

When an individual selects the Set Bolus menu item, the infusion device 10 guides the individual through the steps necessary to select the bolus type (if more than one type is available), enter bolus amount(s), enter bolus duration(s), and initiate delivery, as shown in FIG. 10.

When the Bolus History menu item is selected, the LCD 18 displays a list of previous boluses that have been delivered. In preferred embodiments, the list includes the date and time as well as the amount and type of bolus delivered, as shown in FIG. 9. Preferably, the list is in reverse chronological order starting with the latest bolus delivery at the top of the list. The arrow keys 108 and 112 are used to scroll through the data. In alternative embodiments, the data may be ordered differently, for example in chronological order, or in order of ascending or descending amount, by type, or the like. In further alternative embodiments, more or less data may be available. In still other alternative embodiments, the individual may choose what data to display and/or the order to display it.

When selecting the Max Bolus menu item, the individual may modify the maximum bolus amount that can be delivered in a single bolus. When selecting the Dual/Square Bolus menu item, the individual may turn on or off an option to use a square wave bolus and/or a dual wave bolus type when setting a bolus. This makes more than one bolus type available when the Set Bolus menu item is selected from the Bolus Menu. Finally, the individual may select the Easy Bolus menu item to either turn on or off the option to have an easy bolus (one key used for setting the bolus amount). When the Easy Bolus Option is first turned on, a set screen is displayed to enter an easy bolus amount.

In preferred embodiments, there are other methods of implementing a bolus delivery. For example, from the Blank Screen the individual may press the Up-Arrow key 108 to display the Set Easy Bolus screen, as shown in FIG. 11. Then the individual may use the Up-Arrow key 108 to increase the bolus amount by increments of 0.5 units. When the desired bolus amount is displayed, the individual may activate the bolus delivery by pressing the ACT key 110. In alternative embodiments, the bolus amount may be adjusted by larger or smaller increments. In other alternative embodiments, the bolus amount may be decreased by using the Down-Arrow key 112.

In another example for implementing a bolus delivery, from the Blank Screen the individual may press the Express Bolus key 116, which performs the same function or a similar function as selecting the Set Bolus menu item from the Bolus Menu when the Dual/Square option is turned off, as shown in FIG. 12.

In alternative embodiments, a bolus estimator may be accessed through the menu structure and may be turned on or off by the individual. It is used to estimate the appropriate bolus amount of insulin to control the user's blood glucose level when the user consumes carbohydrates. The bolus estimator is of the type described in U.S. patent application Ser. No. 09/334,858, filed on Jun. 16, 1999, entitled “External Infusion Device with Remote Programming, Bolus Estimator and/or Vibration Alarm Capabilities”; and PCT publication Serial No. US99/18977, filed on Aug. 17, 1999, entitled “External Infusion Device with Remote Programming, Bolus Estimator and/or Vibration Alarm Capabilities,” which are incorporated by reference herein. In particular alternative embodiments, Bolus Est is a menu item in the Bolus Menu. When the individual selects Bolus Est, the infusion device 10 leads the individual through a series of screens to acquire information about the user that affects the bolus estimation calculation such as, the number of grams of carbohydrates to be consumed, the user's current blood glucose level, the desired blood glucose level, the user's insulin sensitivity, the carbohydrate ratio (the number of grams of carbohydrates that is covered by one unit of insulin), and the like. In particular alternative embodiments, the user's insulin sensitivity, desired blood glucose level, and carbohydrate ratio are entered separately and when using the bolus estimator an individual need only enter the grams of carbohydrates to be consumed, and the user's current blood glucose level. In other alternative embodiments, more or fewer inputs are needed. And in further alternative embodiments, the user's current blood glucose level is provided by a blood glucose measurement device.

### Suspend (FIG. 13)

If a bolus or basal delivery is in progress, the individual may choose to suspend fluid delivery. In preferred embodiments, this is done by selecting the Suspend menu item in the Main Menu. A flashing screen prompts the individual to press the ACT key 110 to stop all fluid delivery. If the ACT key 110 is not pressed, the infusion device 10 continues to deliver fluid. The individual may change their mind before pressing the ACT key 110, and instead press the Esc key 114 to return to the Main Menu, or wait for the flashing screen to time-out and return to the Blank Screen or a bolus delivery screen, as shown in FIG. 13. Once the infusion device has been stopped, the individual may press the ACT key 110 twice to restart basal fluid delivery. In alternative embodiments, the fluid delivery is stopped immediately upon selecting the Suspend menu item from the Main Menu. And in other alternative embodiments, basal or bolus delivery amounts must be reentered before fluid delivery can be restarted.

### Basal (FIGS. 14 and 15)

In preferred embodiments, the Basal Menu is displayed when the Basal menu item is selected in the Main Menu. The menu items included in the Basal Menu are: Set/Edit Temp Basal, Set/Edit Basal, Basal Review, Max Basal Rate, and Patterns, as shown in FIG. 14.

When the Set/Edit Temp Basal menu item is selected, a set screen is displayed for the individual to enter a duration followed by another set screen for the individual to enter a basal rate. Once the duration and rate are entered, the individual may press the ACT key 110 to return the Basal Menu and begin delivering fluid at the temporary basal rate. The preexisting basal rate is temporarily overridden. Once the duration has expired, the infusion device 10 returns to delivering fluid according to the preexisting basal rate that was active before the temporary basal rate was begun.

For many users, the required basal rate changes throughout the day. For example, the basal rate required while sleeping may be different from the basal rate needed just before awaking, which may be different from the basal rate needed during an active day. In preferred embodiments, an individual may enter a basal pattern into the infusion device 10 that adjusts the basal rate at various times during the day, or may enter a basal pattern that consists of a single basal rate. Furthermore, for some users the basal pattern needed may vary from one day to another. For example, a different basal pattern may be needed on a day that is filled with strenuous exercise compared to a less physically active day of working at a computer. A standard basal pattern may be needed during weekdays while a different basal pattern is needed for weekends. In particular embodiments, an individual may program and store more than one basal pattern and then select one of the patterns to be active.

A basal pattern consists of a list of basal start times paired with basal rates. Each basal start time represents a time of day that the infusion device 10 will change the basal rate. The infusion device 10 delivers fluid at a basal rate that is paired with the most recent basal start time until a new basal start time is reached, at which time the infusion device 10 changes to the new basal rate associated with the new basal start time. A basal pattern defines the basal rates for an entire 24-hour period. A basal pattern may have only one basal start time and one basal rate (a continuous basal rate all day) or the basal pattern may have many start times each associated with a basal rate (varying basal rates through out the day). In alternative embodiments, basal patterns may be generated for periods longer or shorter than 24 hours.

To enter more than one basal pattern into the infusion device 10, an individual may select Patterns from the Basal Menu, and then select ‘On’ in the Patterns Option screen, as shown in FIG. 14. When the Patterns Option is turned-on, an additional menu item, “Select Patterns,” becomes available in the Basal Menu. If the individual selects the Select Patterns menu item from the Basal Menu, a Select Patterns screen is displayed showing a list of patterns from which the individual may choose. A selected pattern will not be accepted unless a basal pattern has been programmed by using the Set/Edit Basal menu item from the Basal Menu, as described below. When a pattern has been programmed, a value representing the total units delivered in a 24-hour period is displayed next to the pattern name on the Select Pattern screen. If a pattern has not yet been created, the numeric values representing the total units delivered in a 24-hour period are missing, and may be replaced with dashed lines, blank spaces, zeros, and the like.

In preferred embodiments, an individual may select the Set/Edit Basal menu item from the Basal Menu to set a basal rate, edit a basal rate, create new basal pattern, or edit an existing basal pattern, as shown in FIG. 15. If the Patterns Option is on, an Edit Basal screen displays patterns from which the individual may select. The individual may use the arrow keys 108 and 112 to highlight a pattern and then use the ACT key 110 to select the highlighted pattern. Once a pattern has been selected, a screen appears with the title “SET BASAL RATE 1”. Also shown on the screen is a default time, 12:00 A for Start Time 1. In alternative embodiments, other default start times may be used or the individual may enter a time for Start Time 1. In preferred embodiments, the name of the pattern that has been selected appears on the screen, for example, ‘A’ for ‘Pattern A’, and ‘B’ for ‘Pattern B’, and nothing for ‘Pattern Standard’. In alternative embodiments, more or less patterns may be available, and other methods may be used to represent the pattern that is selected such as symbols, numbers, names, days of the week, or the like. Also shown on the display is a value for Basal Rate 1. The value for Basal Rate 1 is flashing to indicate to the user that this value may be modified by pressing the Up or Down Arrow keys 108 and 112. If a basal rate had been previously entered for Basal Rate 1, the screen displays the pre-existing value for the basal rate. Otherwise, the basal rate is displayed as 0.0.

The individual uses the Up or Down Arrow keys 108 or 112 to increment or decrement the flashing basal rate. When the desired basal rate is displayed, the user may press the ACT key 110 to enter the displayed rate for Basal Rate 1 and move on to the next screen. The next screen contains the title “SET START TIME 2”, and the time displayed on the screen is flashing. When no start time has been previously entered for Start Time 2, dashes are used to represent blank spaces into which the time may be entered. In alternative embodiments, when no start time has been previously entered, the default start time is the time for the last start time. For example, if no start time has been entered before for Start Time 3, then the start time for Start Time 2 is the default for Start Time 3. In other alternative embodiments, the default start time is one hour later than the last start time. In preferred embodiments, the individual may use the Up or Down Arrow keys 108 and 112 to increment the start time. When the ACT key 110 is pressed, the start time is entered and the title changes to “SET BASAL RATE 2.” A pre-existing basal rate flashes indicating to the individual that the Up or Down Arrow keys 108 and 112 may be used to change the basal rate. If a value was not previously entered for Basal Rate 2, then the screen displays flashing dashed lines to represent blank spaces that will contain a basal rate once entered. The individual continues to enter additional basal start times and basal rates until they have created their desired pattern. In alternative embodiments, a default value is used instead of dashed lines when no value has been previously entered. In particular alternative embodiments, the default value is the last basal rate before the one that is being programmed. In preferred embodiments, if the individual presses the ACT key 110 when dashed-lines are displayed (whether for a basal start time or a basal rate), the new pattern is considered complete and is entered, and the screen changes to display information about the current basal rate. If at anytime during the entry of a basal pattern the individual presses the Esc key 114, or allows the screen to timeout, the screen changes to display information about the current basal rate and the changes to the basal pattern are not entered. In alternative embodiments, pressing the ACT key 10 when a default value is displayed causes the pattern to be entered and exits the set screen. In further alternative embodiments, other keys are used to indicate that, the pattern is complete.

In preferred embodiments, 1 to 48 different basal start times and basal rates may be entered to create a basal pattern. The start times may be set to begin on any hour or any half-hour. The basal rate resolution is limited to 1/10 of a unit per hour and a maximum basal rate may not be exceeded. In alternative embodiments, more or less start times and basal rates may be used, the start times may be set to any time of day, and/or a finer or courser resolution may be used to set the basal rate.

In preferred embodiments, an individual may review the basal patterns by selecting Basal Review in the Basal Menu. If the Patterns Option is on, a basal review screen will display the various selectable patterns. Once the user selects a pattern, the screen displays a list of each of the start times and the basal rates associated with each of the start times. If the Patterns Option is off, then selecting Basal Review from the Basal Menu immediately displays the ‘Standard’ basal pattern with each of its start times and basal rates.

In preferred embodiments, a maximum basal rate limit may be set to prevent an individual from entering an unintentionally high basal rate. After selecting Max Basal Rate from the Basal Menu, a Max Basal Rate screen displays the pre-existing Max Basal Rate as a flashing value. The Max Basal Rate may be changed by using the Up or Down Arrow keys 108 and 112. Pressing the ACT key 110 enters the new Max Basal Rate. The lowest allowed setting for the Max Basal Rate is the largest basal rate already programmed into an existing basal pattern. In particular embodiments, a value is preprogrammed into the infusion device 10 by the factory or by a health-care professional to limit the maximum setting of the Max Basal Rate limit that maybe entered by an individual. In alternative embodiments, the Max Basal Rate menu item is not available to users.

Priming (FIGS. 16-17(b))

When the reservoir 26 of the infusion device 10 is empty or running low, it may be removed and refilled, or replaced with a filled reservoir. Once the reservoir 26 is replaced, the entire system must be primed so that fluid fills the entire fluid path from the reservoir 26 into a first end of the tubing 28, through the tubing 30, out of the second end of the tubing 31, and through the infusion set 32. And, in preferred embodiments, and individual may select the Prime menu item from the Main Menu. In preferred embodiments, the Prime Menu contains the following menu items: Rewind, Fixed Prime, and Prime History.

When Rewind is selected from the Prime Menu, the information displayed on the infusion device 10 guides the individual through a series of steps to rewind the plunger slider, install a new reservoir, and prime the system, as shown in FIG. 16. First, a rewind message is displayed, telling the user to disconnect the infusion set from the body and then to press the ACT key 110 to rewind the plunger slider. Preferably, the empty reservoir is removed from the infusion device when the infusion set is disconnected from the body. Once the ACT key 110 is pressed, the display shows a message indicating that the plunger slider is rewinding and instructs the user to wait for notification. Once the rewind is complete, a screen is displayed indicating that the rewind is complete so that the individual may install a filled reservoir. Then an Insulin Type screen is displayed with a list of insulin formulation concentrations along with the type of reservoir (pre-filled or user-filled). Once the individual selects the reservoir type along with the insulin type, a confirmation screen displays the selected type for the individual to verify by selecting either ‘Yes’ or ‘No’. In preferred embodiments, the default for the screen is ‘No’. Consequently, the individual must use an arrow key to highlight ‘Yes’ and then use the ACT key 110 in order to verify the insulin type. In alternative embodiments, ‘Yes’ is the default. In other alternative embodiments, no verification is used and/or a key (such as the Esc key 114) may be used to return to a previous screen to change an input. In still other alternative embodiments, the Insulin Type screen is replaced with one or more screens listing other medicaments, treatments, or therapies from which the individual may select. In further alternative embodiments, the reservoir is removed after the plunger slider is rewound.

Continuing with the preferred embodiments, after the insulin type verification, the display shows manual prime instructions, again telling the user to disconnect the infusion set from the body (this is an extra warning incase a user has installed the infusion set before priming), insert and lock the reservoir, and then press the ACT key 110 to prime. When individual presses the ACT key 110, the display shows a screen telling the user to please wait as the infusion device is preparing to prime. The infusion device 10 automatically drives the plunger slider forward until it is engaged with the reservoir piston. Then a priming screen is displayed telling the user to hold the ACT key down to prime the system or to press the Esc key if they are done priming manually. Preferably, the number of units displaced during the priming operation is displayed on the screen. When the individual presses the Esc key 114, the Prime Menu is displayed with an additional menu item, Manual Prime. From the Priming Menu, the individual may select Rewind to rewind the plunger slider again, Manual Prime to manually prime the system by holding down the ACT key 110 again, Fixed Prime to access set screen and enter a number of units for the plunger slider to displace (as shown in FIG. 17(a)), or Prime History to display an information screen containing information from previous primes (as shown in FIG. 17(b)). If any menu item other than Manual Prime is selected, then upon returning to the Prime Menu, Manual Prime will no longer be available as a menu item. This is a safety feature to protect the user from using the Manual Prime feature to infuse fluid into their body. When priming is complete, the display returns to the Blank Screen and the user inserts the infusion set 32 into their body. In alternative embodiments, the display shows a screen telling the user when priming is complete and/or instructing the user to insert the insertion set.

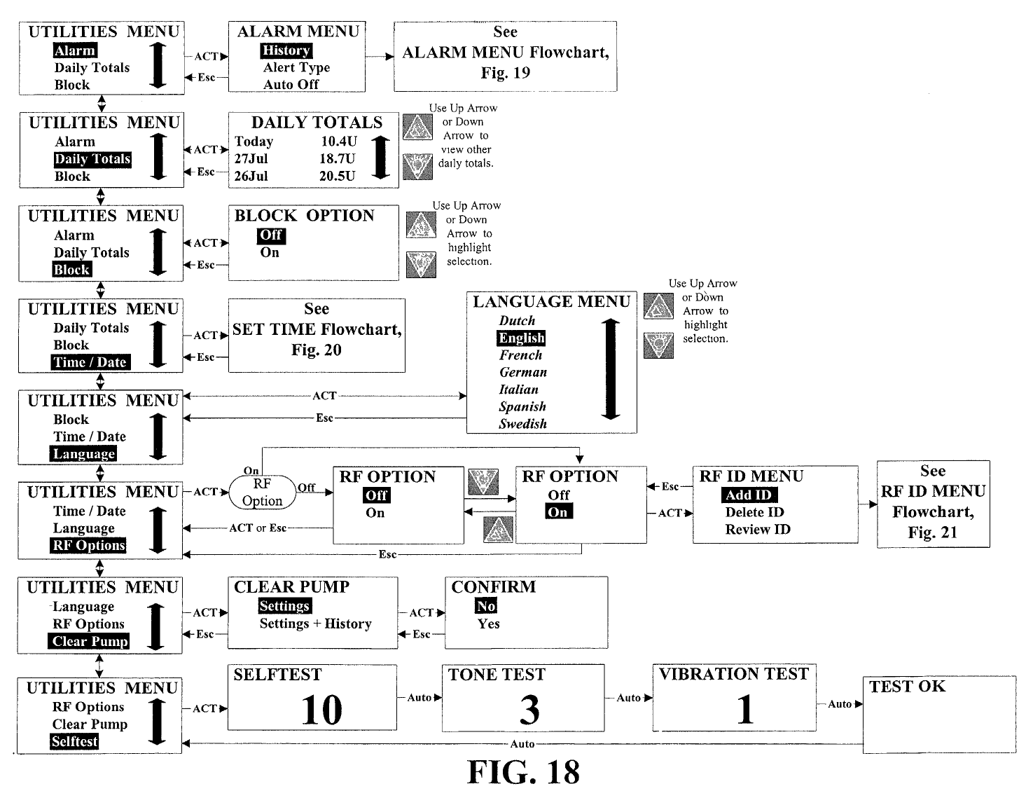

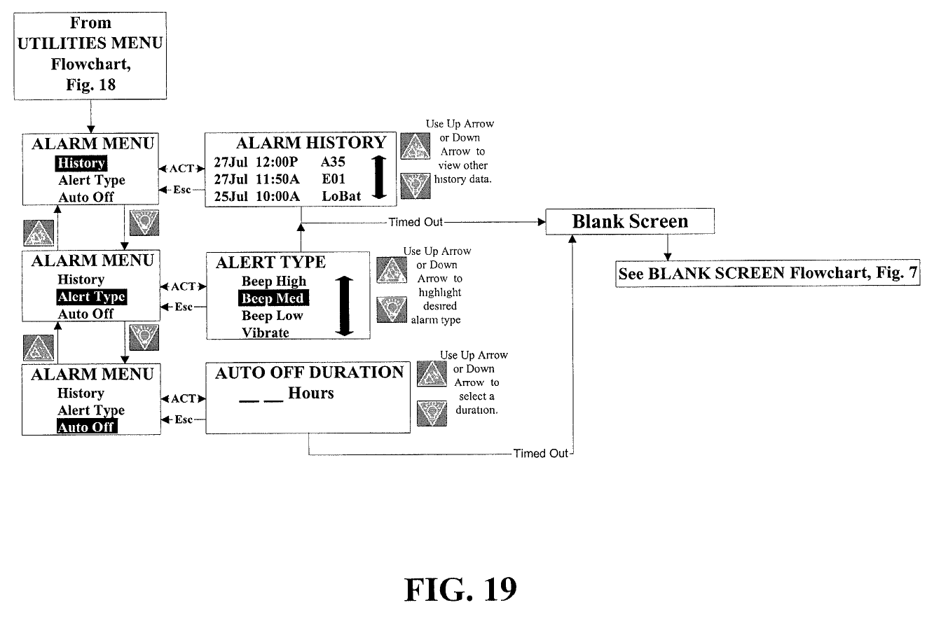

### Utilities (FIGS. 18-21)

In preferred embodiments, miscellaneous setup and maintenance functions are accessible by selecting Utilities from the Main Menu, which brings up a Utilities Menu, as shown in FIG. 18. The Utilities Menu includes the following menu items: Alarm, Daily Totals, Block, Time/Date, Language, RF Options, Clear Pump, and Selftest.

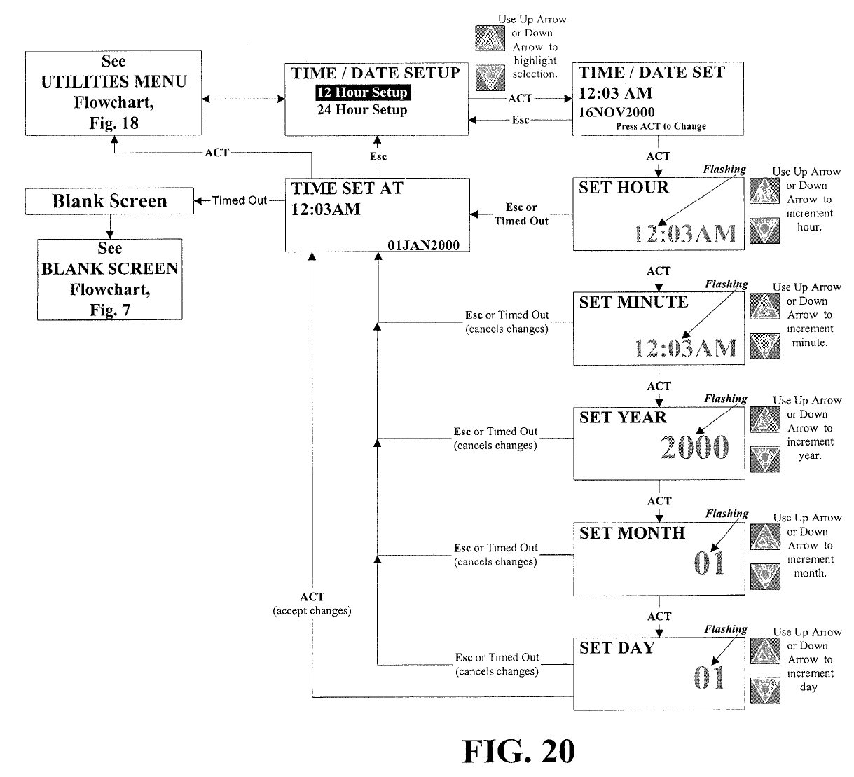

An individual may select Alarm from the Utilities Menu to display an Alarm Menu, which includes three menu items: History, Alert Type, and Auto Off, as shown in FIG. 19. Selecting Alarm History allows an individual to view a screen that lists the date, time, and type of alarms that have been issued by the infusion device 10. Selecting Alert Type brings up a selection screen containing a list of various alert types that an individual may choose for the infusion device 10 to use during an alarm. Alert types from which the individual may choose include, Beep High, Beep Med, Beep Low, and Vibrate. Selecting Auto Off allows an individual to enter a number of hours until the infusion device 10 turns-off. In alternative embodiments, a larger or smaller number of alert types are available. In other alternative embodiments, other types of the alert types are used such as, transmitted messages, lights, flashing LEDs, flashing LCD backlight, Braille messages, electrical scintillation, sounds, vibrations, other types of optics, combinations of alarm types, and the like. Instill other alternative embodiments, the individual may select from various ways for changing the intensity of the alarm when it is not noticed. For example, the individual may select to have an audible alarm increase in volume until responded to. Other types of alarm intensity variation may be selectable as well, such as the methods discussed earlier under hardware embodiments.

In preferred embodiments, Daily Totals may be selected from the Utilities Menu to display a list containing dates and the total number of units delivered for each date.

Selecting Block from the Utilities Menu displays a Block Option screen, which allows an individual to turn on or off the Block Option. Generally, this option is used by parents to prevent children from modifying control parameters on the infusion device 10. When the Block Option is turned-on, all of the select screens and/or set screens that are normally used to change control parameters become inaccessible. In alternative embodiments, an individual may select the individual features to be blocked. For example, the Max bolus and Max basal control parameters may be blocked while still allowing the user access to deliver a bolus or modify a basal pattern. In other alternative embodiments, a password, a code, a series of keystrokes, or the like is used to turn off the Block Option.

Selecting Time/Date from the Utilities Menu gives an individual access to set the time and date for a clock/calendar in the infusion device 10. The individual may select from a 12 hour setup or a 24-hour setup and then may use the arrow keys (108 and 112) and the ACT key 110 to change the hours, minutes, year, month, and day, as shown in FIG. 20.

Selecting Language from the Utilities Menu, displays a Language Menu with a list of Languages from which the individual may choose.

In alternative embodiments, the individual may select or set parameters for the infusion device to accept information from or communicate with other devices such as an RF programmer with a display, blood glucose sensor, blood glucose monitor, blood glucose meter, PDA, and the like.

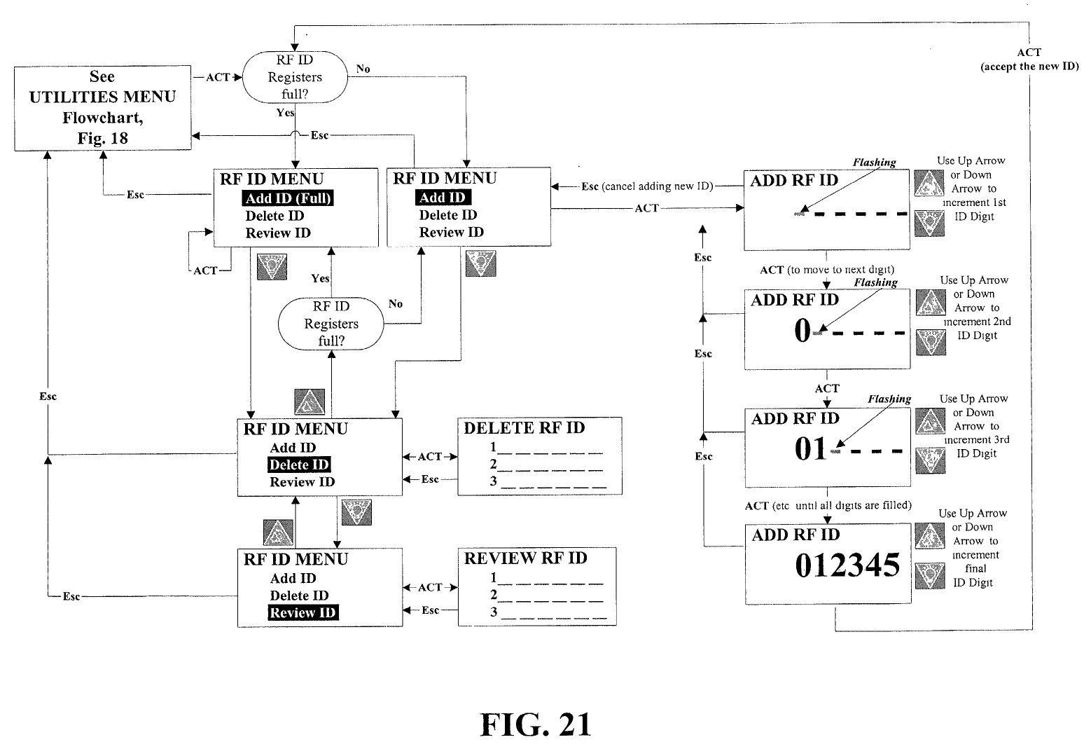

In preferred embodiments, an individual may select RF Options from the Utilities Menu to change the list of RF programmers from which the infusion device 10 will accept information. When the RF Option is turned-on, RF programmers whose ID is stored in the infusion device may communicate with the infusion device 10. An individual may turn-on RF Options by selecting RF Options from the Utilities Menu and then selecting ‘On’. When the RF Option is turned-on, an RF ID Menu displays a list from which the individual may select to Add ID, Delete ID, or Review ID of RF programmers that can communicate with the infusion device 10, as shown in FIG. 21.

In preferred embodiments, the individual may reset the control parameters to factory default values and may clear data from the memory 16. The individual may select Clear Pump from the Utilities Menu to display the Clear Pump screen. Then the individual may select either Settings or Settings+History. A confirmation screen is then displayed, and the individual must use an arrow key 108 or 112 to select ‘Yes’ and then press the ACT key 110 to clear the settings (control parameters) or settings+History (control parameters and data). In alternative embodiments, the individual may reset the control parameters to values set by a health-care professional. In further alternative embodiments, the individual may select between resetting the control parameters to values set by the health-care professional or to factory default values.

In preferred embodiments, an individual may command the infusion device 10 to conduct a self-test by selecting Selftest from the Utilities Menu. In preferred embodiments, a countdown screen is displayed with headings to indicate the progress through stages of the test. In alternative embodiments, other information may be displayed during the selftest such as, diagnostics, bugs, a graphic indicating progress, general pump performance information, time until warrantee expires, maintenance recommendations, a method to contact customer service, and the like.

In alternative embodiments, the infusion device has one or more dedicated keys that act as a short cut for selecting anyone of the menu items in the Main Menu. For example, pressing a particular key causes the Bolus Menu to be displayed. Other keys directly suspend fluid delivery, display the Basal Menu, display the Prime Menu, and/or display the Utility's Menu. Pressing a key has no effect and/or causes a warning message to be displayed if the function represented by the key is inappropriate given the current operation of the infusion device. For example, pressing the suspend key has no effect if the infusion device is not delivering fluid. And the bolus, suspend, or basal keys would have no effect if the infusion device is in a prime mode and a reservoir is not properly installed.

In other alternative embodiments, menu items are in a different order or are located in other menus. The menus and/or menu items may have different names and more or less features may be available.

While, in general, the description of the menu structure above has focused on applications with infusion devices, other embodiments of the invention employ the menu structure to improve programmability of glucose monitors, combined glucose monitor/infusion devices, and/or other programmable medical devices. In alternative embodiments, other menu items may be included such as, glucose alarms and warnings (for setting various limits on glucose measurements), glucose units (for setting the units used to display the blood glucose values), calibration (for conducting blood glucose calibration, reviewing calibration history, calculating the sensor's sensitivity, and the like), glucose history (for reviewing various lists of blood glucose measurements), controller (for turning on or off a closed loop controller, setting controller gains, reviewing controller command history, and the like), and signal processor (for turning on or off one or more filters, setting filer parameters, reviewing raw data, reviewing filtered data, and the like). In further alternative embodiments, the infusion device is capable of storing blood glucose measurements. In particular alternative embodiments, the stored blood glucose measurements may be accessed for viewing through the menu structure.

While the description above refers to particular embodiments of the present invention, it will be understood that many modifications may be made without departing from the spirit thereof. The accompanying claims are intended to cover such modifications as would fall within the true scope and spirit of the present invention.

The presently disclosed embodiments are therefore to be considered in all respects as illustrative and not restrictive, the scope of the invention being indicated by the appended claims, rather than the foregoing description, and all changes which come within the meaning and range of equivalency of the claims are therefore intended to be embraced therein.

1.-46. (canceled)

47. A portable infusion system for infusing insulin into a user, comprising:
a blood glucose meter to communicate blood glucose information; and
an insulin infusion device, including:
a drive mechanism that forces the insulin out of a reservoir;
an input device that accepts one or more inputs;
a processor that uses at least one of the one or more inputs to control the drive mechanism;
a wireless communication device to accept the blood glucose information communicated from the blood glucose meter; and
a display screen to display the blood glucose information, and a screen that allows the user to signify an identity of the blood glucose meter to configure the insulin infusion device to accept communications from the blood glucose meter.

48. The portable infusion system according to claim 47, wherein the insulin infusion device further includes a memory to store the accepted blood glucose information communicated from the blood glucose meter.

49. The portable infusion system according to claim 47, wherein the display screen displays at least one menu item on a menu for selection by the user to display the blood glucose information.

50. The portable infusion system according to claim 47, wherein the insulin infusion device further includes an alarm.

51. The portable infusion system according to claim 47, wherein the insulin infusion device further includes a housing that houses the reservoir, the drive mechanism, the input device, the processor, the wireless communication device, and the display screen.

52. The portable infusion system according to claim 47, further including an infusion set and tubing having a first end and a second end, wherein the first end of the tubing is connected to the reservoir and the second end of the tubing is connected to the infusion set.

53. The portable infusion system according to claim 47, wherein the input device includes a keypad with one or more keys.

54. A portable infusion system for infusing insulin into a user, comprising:
a blood glucose meter to communicate blood glucose information; and
an insulin infusion device, including:
a drive mechanism that forces the insulin out of a reservoir;
an input device that accepts one or more inputs;
a processor that uses at least one of the one or more inputs to control the drive mechanism;
a wireless communication device to accept the blood glucose information communicated from the blood glucose meter;
a bolus estimator to estimate an appropriate bolus amount of insulin to be delivered into the user based upon the accepted blood glucose information communicated from the blood glucose meter; and
a display screen to display the blood glucose information and a menu to access the bolus estimator.

55. The portable infusion system according to claim 54, wherein the insulin infusion device further includes a memory to store the accepted blood glucose information communicated from the blood glucose meter.

56. The portable infusion system according to claim 54, wherein the display screen displays at least one menu item on the menu for selection by the user to display the blood glucose information.

57. The portable infusion system according to claim 54, wherein the insulin infusion device further includes an alarm.

58. The portable infusion system according to claim 54, wherein the insulin infusion device further includes a housing that houses the reservoir, the drive mechanism, the input device, the processor, the wireless communication device, and the display screen.

59. The portable infusion system according to claim 54, further including an infusion set and tubing having a first end and a second end, wherein the first end of the tubing is connected to the reservoir and the second end of the tubing is connected to the infusion set.

60. The portable infusion system according to claim 54, wherein the appropriate bolus amount of insulin to be delivered into the user is further based upon at least one of: a desired blood glucose level, a number of grams of carbohydrates to be consumed, the user's insulin sensitivity, and a carbohydrate ratio of the user.

61. A portable infusion system for infusing insulin into a user, comprising:
a blood glucose meter to communicate blood glucose information; and
an insulin infusion device, including:
a drive mechanism that forces the insulin out of a reservoir;
an input device that accepts one or more inputs;
a processor that uses at least one of the one or more inputs to control the drive mechanism;
a wireless communication device to accept the blood glucose information communicated from the blood glucose meter; and
a display screen to display the blood glucose information.

62. The portable infusion system according to claim 61, wherein the insulin infusion device further includes a memory to store the accepted blood glucose information communicated from the blood glucose meter.

63. The portable infusion system according to claim 61, wherein the display screen displays at least one menu item on a menu for selection by the user to display the blood glucose information.

64. The portable infusion system according to claim 61, wherein the insulin infusion device further includes an alarm.

65. The portable infusion system according to claim 61, wherein the insulin infusion device further includes a housing that houses the reservoir, the drive mechanism, the input device, the processor, the wireless communication device, and the display screen.

66. The portable infusion system according to claim 61, further including an infusion set and tubing having a first end and a second end, wherein the first end of the tubing is connected to the reservoir and the second end of the tubing is connected to the infusion set.
